SEQUENCE LISTING (15 sequences)

SEQ ID NO: 1 - protein, 597 aa, Mus musculus
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLVDQTSGDQSPLPPCTPTPPCAEMREDSARVYENVGLMQQQRSFR

SEQ ID NO: 2 - protein, 593 aa, Homo sapiens [sequence omitted]

SEQ ID NO: 3 - protein, 99 aa, Homo sapiens
RWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLN

SEQ ID NO: 4 - protein, 389 aa, Homo sapiens
TLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 5 - protein, 11 aa, Homo sapiens
VHCSAGIGRTG

SEQ ID NO: 6 - protein, 594 aa, Homo sapiens
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKSLKRK

SEQ ID NO: 7 - protein, 611 aa, Rattus norvegicus
MLSRGWFHRDLSGPDAETLLKGRGVPGSFLARPSRKNQGDFSLSVRVDDQVTHIRIQNSGDFYDLYGGEKFATSTELVEYYTQQQGILQDRDGTIIHLKYPLNCSDPTSERWYHGHMSGGQAESLLQAKGEPWTFLVRESLSQPGDFVLSVLNDQPKAPGSPLRVTHIKVMCEGGRYTVGGSETFDSLTDLVEHFKKTGIEEASGAFVYLRQPYYATRVNAADIENRVLELNKKQESEDTAKAGFWEEFESLQKQEAKNLHQRLEGQRPENKSKNRYKNILPFDHSRVILQGRDSNIPGSDYINANYVKNQLLGPDENSKTYIASQGCLDATVNDFWQMAWQENTRVIVMTTREVEKGRNKCVPYWPEVGTQRVYGLYSVTNCKEHDTAEYKLRTLQISPLDNGDLVREIWHYQYLSWPDHGVPSEPGGVLSFLDQINQRQESLPHAGPIIVHCSAGIGRTGTIIVIDMLMESVSTKGLDCDIDIQKTIQMVRAQRSGMVQTEAQYKFIYVAIAQFIETTKKKLEIIQSQRGQESEYGNITYPPALRSAHAKASRTSSKHKEEVYENVHSKNKKEEKVKKQRSADKEKNKGSLKRISLTPCRGLRWADRDL

SEQ ID NO: 8 - protein, 598 aa, Mus musculus
MLSRGWFHRDLSGPDAETLLKGRGVPGSFLARPSRKNQGDFSLSVRVDDQVTHIRIQNSGDFYDLYGGEKFATLTELVEYYTQQQGILQDRDGTIIHLKYPLNCSDPTSERWYHGHISGGQAESLLQAKGEPWTFLVRESLSQPGDFVLSVLNDQPKAGPGSPLRVTHIKVMCEGGRYTVGGSETFDSLTDLVEHFKKTGIEEASGAFVYLRQPYYATRVNAADIENRVLELNKKQESEDTAKAGFWEEFESLQKQEVKNLHQRLEGQRPENKSKNRYKNILPFDHHSRVILQGRDSNIPGSDYINANYVQNQLLGPDENSKTYIASQGCLDATVNDFWQMAWQENTRVIVMTTREVEKGRNKCVPYWPEVGTQRVYGLYSVTNSREHDTAEYKLRTLQISPLDNGDLVREIWHYQYLSWPDHGVPSEPGGVLSFLDQINQRQESLPHAGPIIVHCSAGIGRTGTIIVIDMLMESISTKGLDCDIDIQKTIQMVRAQRSGMVQTEAQYKFIYVAIAQFIETTKKKLEIIQSQKGQESEYGNITYPPAVRSAHAKASRTSSKHKEEVYENVHSKSKKEEKVKKQRSADKEKNKGSLKRK

SEQ ID NO: 9 - protein, 582 aa, Oncorhynchus gorbuscha
MVRWFHRDISGLDAESVLKGRGVHGSFLARPSRKNQGDFSLSVRVGELVTHIRIQNTGDFYDLYGGEKFATLSELVEYYTAENGILQDKDGTIIELKYPFNCSDPTTERWYHGHLSGPNAEKLLWERDEPGTFLVRESLSKPGDFVLSVLTEEKSKASSGGRRVSHIKIMCQNDRYTVGGKEMFDTLADLMEHYKRKGIEEMSGTWVHLKQPYFSTRVNAADIDSRVRLLDQMAEGENEGDKKSKAGFWEEFDALQKQETKVKKSREEGMRPENKSKNRYKNILPFDETRVILSSGDPNIIGSDYINGNYVTNKLQEPGDQKVYIACQGCLATTVNDFWQMVWQERTRVIVMTTREVEKGRNKCVPYWPEMQGSKEVGPYVVTCVSERDATDYKIRVMEISPLDQSDSVRTIWHYQYLSWPDHGVPEEPGGVLSFLSQVNLKQAEFTNAGPMIIHCSAGIGRTGTIVVIDMIIETIDTKGLDCDIDIQKSIQMVRDQRSGMVQTEAQYKFIYLAVSEYIEASKTYNKGAETEYGNLQFKHQPASCKVSKNKEQQLYENIAKGKKDVKKQKSEEKKSGSVRKR

SEQ ID NO: 10 - protein, 597 aa, Mus musculus
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLVDQTSGDQSPLPPCTPTTPCAEMREDSARVYENVGLMQQQRSFR

SEQ ID NO: 11 - protein, 597 aa, Rattus norvegicus
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDSKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDECALKEYGVMRVRNVRESAAHDYTLRELKLSKVGQALLQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGNVQTEAQYRFIYMAVQHYIETLQRRIEEEQKSKRKGHEYTNIKSSLVDQTSGDQSPLPPCTPTPPCAEMREDSARVYENVGLMQQQRSFR

SEQ ID NO: 12 - protein, 597 aa, Homo sapiens
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKGHSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRNVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGNVQTEAQYRPIYMAVQHYIETLQRRIEEEQKSKRKGHEYTNIKYSLADQTSGDQSPLPPCTPTPPCAEMREDSARVYENVGLMQQQKSFR

SEQ ID NO: 13 - protein, 595 aa, Xenopus laevis
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKTESNDGKSKVTHVMIRCQDIKYDVGGGEKFDSLTDLVEHYKKNPMVETLGTVLQLKQPLNTTRINAAEIEIRVKELSKPAETADKFKQGFWEEFETLQQQECKLLYSRKEGQRPENKNKNRYKNILPFDHTRVELHDGDPNEQVSDYINANIIMPEFEIKCINPKPKKRYIATQGCLQNTVNDFWRMVFQENSRVIVMTTKEVERGKSKCVKYWPDEFALKEYGVMRVRNVKETPAHDYILRELKLSKVGQGNTERAVWQYHFKTWPDHGVPADPGGVLDFLEEVHHKQDGITDAGPVVVHCSAGIGRTGTFIVIDILIDVIREKGVDCDIDVPKTIQMVRSQRSGMVQTEAQYRFIYMAVQHYIETLQRRIEEEQKSKRKGHEYTNIKYSLSEQGDQSPLPPCTPTPLPTPTLPEMREDAGRVYENVGLLQQQKSFR

SEQ ID NO: 14 - protein, 582 aa, Drosophila melanogaster
MSSRRWFHPTISGIEAEKLLQEQGFDGSFLARLSSSNPGAFTLSVRRGNEVTHIKIQNNGDFFDLYGGEKFATLPELVQYYMENGELKEKNGQAIELKQPLICAEPTTERWFHGNLSGKEAEKLILERGKNGSFLVRESQSKPGDFVLSVRTDDKVTHVMIRWQDKKYDVGGGESFGTLSELIDHYKRMPHVETCGTVVHLRQPFNATRITAAGINARVEQLVKGGFWEEFESLQQDSRDTFSRNEGYKQENRLKNRYRNILPYDHTRVKLLDVEHSVAGAEYINANYIRLPTDGREMFKTYIATQGCLLTQQVNTVTDFWNMVWQENTRVIVMTTKEYERGKEKCARYWPDEGRSEQFGHARIQCVSENSTSDYTLREFLVSWRDQPARRIFHYHFQVWPDHGVPADPGCVLNFLQDVNTRQSHLAQAGEKPGPICVHCSAGIGRTGTFIVIDMILDQIVRNGLDTEIDIQRTIQMVRSQRSGLVQTEAQYKPVYAVQHYIQTLIARKRAEEQSLQVGREYTNIKYTGEIGNDSQRSPLPPAISSISLVPSKTPLTPTSADLGTGMGLSMGVGMGVGNKHA

SEQ ID NO: 15 - protein, 631 aa, Caenorhabditis elegans
MPRLALRQYNFYYRVNGEKAEELLKEYGEDGDFLLRYSESNPQNFSISVRVAEDKILHIKVTKYESDMLSIFEDERTTPNQFGSITELAEFYMEFPEKLREKNGLFLELKKPVYVPYHLEACAEEQRRTQLYRWWHGNLPASSANKLLQTEKNGTYLLRASQHIPGALVISAKTEGQVVHLTIYQDPSTGRFNIDGDRTKFQSAWLLIDSYKNPIVEKGEASRVLYLEEPLFNTFIEADLFVDRFEIIRRPINPRESMEKTGISEEFDRLSQEALPAEQYLSKREGRRPVNAEKNRYKNIVPFDHTRVILTDRPNTPGSDYINASYVRFENSQRTKNVTFACEKSFIATQGCLETTISDFWSNVWQENSRVIVMPTMENERKEKCARYWPAEVNKPEVHCDISLTCTIERKVQRAVSDEVKAELEQEREIRQLQYLTWPDHGCPLHPYAVLNFLEDVDREYDYFNAQPIAASLPQGPIVVHCSAGIGRTGTVLVLDALLNQVKKVGLLCPNDVYKMVKYVRTYRSGLVQTEQQYQFLYKALAFYLKNNNPYPVKSFIDGDTDAFDFPRRLRPTPNASRPSSARQVTSSRPSSSASSRTSHSRPRTGPQAEPIFERSTSSTSSSTLLKSTKK

(19) United States
(12) Patent Application Publication
(10) Pub. No.: US 2015/0352131 A1
(43) Pub. Date: Dec. 10, 2015

(54) Compositions and Methods for the Prevention and Treatment of Osteolysis and Osteoporosis

(71) Applicant: RHODE ISLAND HOSPITAL, Providence,, RI (US)

(72) Inventors: Wentian Yang, Pawtucket, RI (US); Michael G. Ehrlich, Providence, RI (US); Yi Zhou, Sharon, MA (US); Douglas Moore, Barrington, RI (US)

(73) Assignee: RHODE ISLAND HOSPITAL, Providence, RI (US)

(21) Appl. No.: 14/760,413

(22) PCT Filed: Jan. 16, 2014
(86) PCT No.: PCT/US2014/011883

§ 371 Date: Jul. 10, 2015

Related U.S. Application Data
(60) Provisional application No. 61/753,218, filed on Jan. 16, 2013.
(60) Provisional application No. 61/849,031, filed on Jan. 16, 2013.

(51) Int. Cl. A61K 31/655; A61L 27/54; A61L 27/28; A61L 26/00; G01N 33/50; A61K 31/4152; A61K 31/192; A61K 31/225; G01N 33/573; A61K 31/663; A61K 31/404

(52) U.S. Cl. CPC A61K 31/655; A61K 31/663; A61L 27/54; A61L 27/28; A61L 26/0066; A61K 31/404; A61K 31/4152; A61K 31/192; A61K 31/225; G01N 33/573; G01N 33/5044; A61L 2430/24; A61L 2430/02; A61L 2420/00; A61L 2300/606; A61L 2300/434; A61L 2300/412; G01N 2500/10; G01N 2333/916

(57) ABSTRACT

This application discloses compositions, devices, and methods for the prevention and treatment of osteolysis and osteoporosis. Treatment or prevention of osteolysis or osteoporosis is carried out by targeting the enzyme, Shp2 (a Src homology 2 (SH2) domain containing non-transmembrane Protein Tyrosine Phosphatase (PTP)), or proteins involved in the Shp2 signaling pathway by administering a Shp2 pathway inhibitor that inhibits fusion of pre-osteoclasts.

### RELATED APPLICATIONS

This application claims the benefit of, and priority to, U.S. Provisional Application No. 61/753,218, filed Jan. 16, 2013, and U.S. Provisional Application No. 61/849,031, filed Jan. 16, 2013. The contents of each of these applications are incorporated herein by reference in their entireties.

### STATEMENT AS TO FEDERALLY SPONSORED RESEARCH

This invention was made with Government support under National Institutes of Health (NIH) grant NIHR21AR057156. The Government has certain rights in the invention.

### FIELD OF THE DISCLOSURE

The present invention relates to orthopedic therapies and devices.

### BACKGROUND OF THE DISCLOSURE

Osteoporosis (OP) and periprosthetic osteolysis (PO) are skeletal disorders causing major health and economic burdens worldwide. According to the NIH, roughly half of all women over age 50 will experience an osteoporosis-related bone fracture. About 10 to 20% of total joint arthroplasty patients suffer from PO and require revision procedures. The drugs currently available to treat OP and PO have low efficacy and unpleasant side effects. Thus, there is a need for safer and more efficacious therapies for PO and OP.

### SUMMARY OF THE DISCLOSURE

The present invention relates to the treatment or prevention of osteolysis or osteoporosis by targeting the enzyme, Shp2 (a Src homology 2 (SH2) domain containing non-transmembrane PTP), or proteins involved in the Shp2 signaling pathway.

The invention features a method of treating or preventing osteolysis or osteoporosis, involving identifying a subject with osteolysis or osteoporosis or at risk for developing osteolysis or osteoporosis, and administering to said subject a Shp2 pathway inhibitor, where the Shp2 pathway inhibitor inhibits fusion of pre-osteoclasts.

The invention also features a composition or pharmaceutical composition comprising a Shp2 pathway inhibitor.

In another aspect, the invention features a device comprising an implant (e.g., orthopedic/dental implant) and a composition of the invention. For example, the device comprises an orthopedic/dental implant and a Shp2 pathway inhibitor, where the Shp2 pathway inhibitor is incorporated into, coupled to, coated onto, or eluted from the implant. Exemplary orthopedic implants are bone screws, orthopedic pins, mechanical devices for the fixation and stabilization of an orthopedic fracture, bone cement, artificial joints, hip replacement joints, joint implants, hip replacements, knee replacements, elbow replacements, synthetic joints, synthetic cartilage, synthetic spin discs, bone plates, orthopedic nails, orthopedic rods, orthopedic rectangles, compression plates, shoulder replacements, bone wires, and prostheses. For example, a dental implant is a replacement tooth root, e.g., that connects directly to a jaw bone. In some examples, a dental implant is an endosteal (in the bone) implant or a subperiosteal (on the bone) implant. Exemplary dental implants include screws, cylinders or blades surgically placed into the jawbone. In some embodiments, each implant holds at least one tooth (e.g., prosthetic tooth). Subperiosteal implants are placed on top of the jaw, and the implant framework contains posts that protrude through the gum of the subject in order to secure the implant. For example, the implant (e.g., orthopedic or dental) comprises a metal (e.g., tantalum, titanium, a titanium alloy such as Ti6Al4V, cobalt, chromium, a cobalt-chromium alloy, zirconium, or a zirconium alloy such as oxinium oxidized zirconium), a ceramic, hydroxyapatite, and/or polyethylene.

In one aspect, the Shp2 pathway inhibitor comprises a Shp2 inhibitor. For example, the Shp2 inhibitor binds to an amino acid comprising the Shp2 catalytic site. In one embodiment, the Shp2 inhibitor binds to an amino acid of the protein tyrosine phosphatase (PTP) domain of Shp2 or a fragment thereof. In some cases, an Shp2 inhibitor binds to the N-terminal Src homology 2 (N—SH2) domain of Shp2 or a fragment thereof. In other examples, the Shp2 inhibitor binds to one or more of residues of the phosphotyrosine binding pocket of Shp2 (e.g., 13, 32, 53, and 55 of human Shp2 (as shown in SEQ ID NO: 2)). In some embodiments, the inhibitor binds to one or more amino acids of a PTP motif of Shp2, e.g., amino acid residues 457-467 of human Shp2, VHCSAGIGRTG (SEQ ID NO: 5). In another example, the Shp2 inhibitor binds to one or more amino acids in human Shp2, where the amino acids are selected from the group consisting of K280, Y279, N280, R362, K364, K366, W423, P424, D425, H426, G427, S460, A461, I463, G464, R465, and Q510, or a homologous residue thereof in a non-human Shp2. A homologous residue in a non-human Shp2 is a residue that aligns in a multiple sequence alignment with a corresponding residue in human Shp2.

In another aspect, the Shp2 inhibitor prevents binding of Shp2 to a binding partner, e.g., GRB2-associated-binding protein 1 (Gab1) or GRB2-associated-binding protein 2 (Gab2).

Exemplary Shp2 inhibitors are NSC 87877, SPI-112, SPI-112Me, PHPS1, SHP2 inhibitor II-B08, C21, tautomycetin, TTN D-1,7-deshydroxypyrogallin-4-carboxylic acid (DCA), NSC-117199, 8Z,11Z-Feptadecadienoic acid, 14Z,17Z-tricosadienoic acid, caffeic acid, 2-hydroxy-3-[(1-oxododecyl)oxy]propyl-β-d-glucopyranoside, and derivatives of the Shp2 inhibitors described herein.

Preferably, the Shp2 inhibitor comprises NSC 87877. The chemical structure of NSC-87877 is shown below.

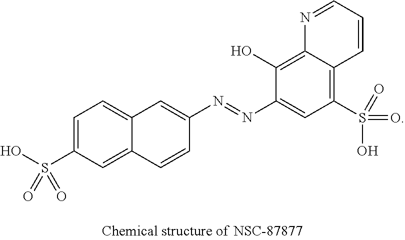 [SMILES: O=S(=O)(O)c1ccc2cc(/N=N/c3cc(S(=O)(=O)O)c4cccnc4c3O)ccc2c1]

In another embodiment, the Shp2 pathway inhibitor comprises an inhibitor of a protein selected from the group consisting of Gab2, Nuclear factor of activated T-cells, cytoplasmic 1 (Nfatc1), Calcitonin receptor, cathepsin K (Ctsk), dendritic cell-specific transmembrane protein (DC-STAMP), Matrix metallopeptidase 9 (Mmp9), and Tartrate-resistant acid phosphatase (Trap).

In certain embodiments, the compositions and devices of the present invention further comprise a bisphosphonate compound. Exemplary bisphosphonates are Aldronate, Etidronate, Clodronate, Tiludronate, Pamidronate, Neridronate, Olpadronate, Alendronate, Ibandronate, Risedronate, and Zoldronate. Preferably, the bisphosphonate comprises Aldronate. In some cases, the effect of a bisphosphonate in combination with a Shp2 pathway inhibitor is synergistic.

The invention also relates to a method of treating or preventing osteolysis or osteoporosis by administering to a subject the compositions or devices described herein. Administration of the compositions or devices of the invention is performed by procedures including but not limited to injection, endoscopic delivery, minimally invasive surgery, arthroscopy, infusion, or surgical implantation. Routes of administration include oral, pulmonary, rectal, parenteral, intradermal, transdermal, topical, transmucosal, subcutaneous, intravenous, intramuscular, intraperitoneal, intratympanic, inhalational, buccal, sublingual, intrapleural, intracerebroventricular (ICV), intrathecal, intranasal, and the like.

Methods of the present invention deliver the compositions described herein to a target tissue (e.g., a specific bone, cartilage, joint, prosthesis, implant, or tissue in proximity to a prosthesis or implant). Alternatively, the compositions described herein are delivered systemically (e.g., oral, intravenous, intramuscular).

The invention also features a method for identifying a candidate compound or molecule for inhibiting osteoclastogenesis. The method comprises contacting a cell expressing an osteoclast precursor with a candidate compound and then measuring the level of Shp2 activity in the cell. A decrease in the level of Shp2 activity in the presence of the candidate compound or molecule compared to that in the absence of the compound or molecule indicates that said compound inhibits osteoclastogenesis. Other methods of screening involve further testing a candidate Shp2 inhibitory compound or molecule in the osteoclast culture system and measuring the number or density of mature osteoclasts after culturing precursors in the presence and absence of the candidate compound or molecule. A decrease in the number or density of osteoclasts in the presence of the compound compared to that in its absence indicated that the compound or molecule inhibits osteoclastogenesis. Alternatively or in addition, the method for identifying a candidate compound for inhibiting osteoclastogenesis comprises contacting a cell expressing an osteoclast precursor cell with a candidate compound in culture and measuring the number of osteoclasts in the culture. A decrease in the number of osteoclasts in the presence of said compound compared to that in the absence of said compound indicates that the compound inhibits osteoclastogenesis.

The invention also provides a method of improving fixation of an implant (e.g., orthopedic/dental implant), comprising administering a Shp2 pathway inhibitor to a subject before, concurrently with, and/or after insertion of the implant. For example, the method comprises administering a Shp2 pathway inhibitor to a subject one week before insertion of the implant. The method comprises administering a therapeutically effective dosage of a Shp2 pathway inhibitor to a subject at the time of surgery and until bone healing is substantially complete (e.g., for 1, 2, 3, 4, 5, 6, 7, 8, 9, 10, 11, 12, 13, 14, 15, 16, 17, or 18 weeks after insertion of the implant), followed by gradually decreasing the dosage of the Shp2 pathway inhibitor, and cessation of the administration of the Shp2 pathway inhibitor. Administration is systemic (e.g., oral, intravenous, intra-arterial, or intramuscular) or local (e.g., injection or infusion).

A Shp2 inhibitor is a small molecule, polypeptide or derivative thereof, antibody or fragment thereof, or nucleic acid (e.g., siRNA, shRNA, PNA, RNA, DNA, or derivative thereof) that reduces the expression of or decreases the catalytic activity of Shp2. Alternatively or concurrently, a Shp2 inhibitor inhibits the binding of Shp2 to a binding partner(s) (e.g., Gab 1 or Gab2). In one aspect, the inhibitor inhibits osteoclast formation. A Shp2 pathway inhibitor is a small molecule, polypeptide or derivative thereof, antibody or fragment thereof, or nucleic acid (e.g., small interfering RNA (siRNA), short hairpin RNA (shRNA), peptide nucleic acid (PNA), RNA, DNA, or derivative thereof) that reduces the expression of or reduces the activity of one or more proteins in the Shp2 signaling pathway involving the protein, Receptor activator of nuclear factor kappa-B ligand (RANKL). The inhibitor inhibits osteoclast formation. In one aspect, the inhibitor decreases the expression (at the protein or nucleic acid, e.g., messenger RNA level) of or activity of Nfatc1, Nfatc2, FBJ Murine Osteosarcoma Viral Oncogene Homolog (c-Fos), MMP9, Trap, or Ctsk, calcitonin receptor, DC-STAMP, Gab1, Gab2, e.g., in bone marrow derived macrophages or osteoclast precursor cells. In some embodiments, the protein or nucleic acid (e.g., mRNA) expression level is reduced by at least 1.5-fold (e.g., at least 1.5-fold, 2-fold, 3-fold, 4-fold, 5-fold, 10-fold, 15-fold, 20-fold, 25-fold, 30-fold, 50-fold, 100-fold, 200-fold, 500-fold, 1000-fold, or more) compared to the expression level of the protein or nucleic acid prior to administration of the inhibitor in a subject.

In another aspect, the inhibitor inhibits binding of one protein to another in the Shp2 signaling pathway. In yet another aspect, the inhibitor inhibits osteoclast formation. In some cases, the Shp2 pathway inhibitor reduces resorption of a bony tissue in the subject. For example, the inhibitor reduces resorption of a bony tissue in a subject, e.g., by at least 1.5-fold, 2-fold, 3-fold, 4-fold, 5-fold, 6-fold, 10-fold, 15-fold, 20-fold, 50-fold, 100-fold, or more compared to the extent of resorption of a subject that has not been administered the inhibitor. In other examples, the inhibitor reduces resorption of a bony tissue at a site of a subject, e.g., by at least 1.5-fold, 2-fold, 3-fold, 4-fold, 5-fold, 6-fold, 10-fold, 15-fold, 20-fold, 50-fold, 100-fold, or more compared to the extent of resorption of a bony tissue at a different site of the same subject. A bony tissue is a type of tissue that is formed by osteoblasts, which deposit calcium, phosphate, and magnesium, along with collagen, to form a crystalline bone mineral. A small molecule is a low molecular weight compound of less than 1000 Daltons, less than 800 Daltons, or less than 500 Daltons.

The invention also provides a use of a composition or device described herein in a method to treat or prevent osteolysis or osteoporosis, wherein the Shp2 pathway inhibitor inhibits fusion of pre-osteoclasts. In addition, the invention provides a use of a composition or device described herein in a method of manufacturing a medicament for the treatment or prevention of osteolysis or osteoporosis, wherein the Shp2 pathway inhibitor inhibits fusion of pre-osteoclasts.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. GenBank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIGS. 1A-D are a series of images and graphs that illustrate that mice lacking Shp2 in osteoclasts are osteopetrotic. FIG. 1A is a schematic of mouse breeding schemes (left) used to generate Shp2fl/+;Ctsk-Cre (Control) and Shp2fl.fl;Ctsk-Cre (KO) mice, and a Western blot (right) confirming reduced expression of Shp2 in bone marrow-derived osteoclasts from KO mice (Extracellular signal-regulated kinase 2 (Erk2) served as a loading control). FIG. 1B is a series of gross images demonstrating the effect of Shp2 deletion in osteoclasts on skeletal development. Shp2 mutant mice had enlarged femurs, tibiae, vertebrae, and humerii. Ectopic bony nodules were also found on the skeletons from Shp2 mutant mice. FIG. 1C is a series of images depicting μ-CT imaging analysis and Masson Trichrome staining that demonstrate the increase in volumetric density and trabeculae in mice with osteoclast-specific Shp2 deletion, as well as decreased trabecular thickness and spacing. Data were collected from the distal femur of 8-week-old Control and KO mice (n=4). BV: bone volume; TV: total volume; Tb.N: trabecular bone number; Tb.Th: trabedular bone thickness; Tb.Sp: trabecular space. FIG. 1D is a series of bar graphs presenting the data from FIG. 1C.

FIGS. 2A-E are a series of images and graphs that illustrate that Shp2 deficiency impairs osteoclastogenesis in vivo and ex vivo. FIG. 2A is a set of images showing TRAP (Tartrate resistant acid phosphatase) staining that reveals the presence of multinucleated osteoclasts (red) on the surface of proximal tibia trabeculae in Control mice but absence of similar cells in KO mice. Slides were counterstained with hemotoxylin (blue). FIG. 2B is a bar graph that presents Histomophometry data showing the average number of osteoclasts/mm² on trabecular bone surface. n=3. FIG. 2C is a set of images of TRAP staining showing the reduced formation of multinucleated osteoclasts in an ex vivo osteoclast culture of isolated bone marrow cells from KO but not Control mice. Bone marrow cells (1×10⁵) were cultured in osteoclast medium as described in the method section. FIG. 2D is a bar graph showing quantification of multinucleated osteoclasts (>3 nucleus) n=4. *p<0.05 (student t test). FIG. 2E is a set of fluorescence microscopic images showing that Shp2 deletion in osteoclasts inhibited new bone formation in KO compared to Control mice in vivo.

FIGS. 3A-E are a series of bar graphs and images that illustrate that Shp2 is required for osteoclast maturation by promoting pre-osteoclast fusion. FIGS. 3A-B are a set of bar graphs presenting data from water soluble tetrazolium salt 1 (WST1) assays showing that bone marrow cells from Control and KO mice have comparable viability and proliferation when cultured for 5 days with either the optimal doses of M-CSF and variable doses of RANKL or the optimal dose of RANKL and variable doses of M-CSF as indicated. The bar graphs show the numbers of viable cells measured by the WST-1 optical density determined using a spectrophotometer at the 420 nm wavelength. FIG. 3C is a series of TRAP staining images of multinucleated osteoclasts cultured under the same condition as described in FIG. 3A and demonstrates the similar numbers of TRAP-positive cells that appear between Control and KO mice, consistent with the results of WST-1 assay shown in FIG. 3A. Increased RANKL concentration in the culture medium with a constant dose of M-CSF promotes the formation of multinucleated osteoclasts (arrow) only in the marrow cells of Control but not KO mice. FIG. 3D is a series of TRAP staining images of multinucleated osteoclasts cultured under the same conditions described in FIG. 3B that demonstrates that similar numbers of cells exist between Control and KO mice when both RANKL and M-CSF doses are high but numbers of cells decline when doses of M-CSF decrease in culture (consistent with the results of WST-1 assay shown in FIG. 3B). Note that multinucleated osteoclasts again only formed in marrow cells from Control but not KO mice under the optimal osteoclast culture condition. FIG. 3E is a set of fluorescence microscopic images showing the formation of multinucleated osteoclasts (red) in an ex vivo osteoclast culture assay from marrow cells of Shp2fl/fl;mTG mice. In contrast, bone marrow-derived cells from KO;mTG mice switch from red to green (indicating Shp2 was deleted in these cells via Ctsk-Cre) and failed to form multinucleated osteoclasts. Blue dots are nuclei of cells stained with 4′,6-diamidino-2-phenylindole (DAPI). All macrophage colony-stimulating factor (M-CSF) and RANKL concentrations are in ng/mL.

FIGS. 4A-D are a set of graphs and images illustrating that the RANK/Shp2/Nfatc1 signaling axis regulates pre-osteoclast fusion. FIGS. 4A-B are a set of bar graphs depicting quantitative PCR analysis of osteoclastogenic gene expression in bone marrow cells from Control and KO mice cultured in osteoclast media. Shp2 deletion inhibits the expression of calcitonin receptor, cathepsin K, Mmp9, Trap, and the two key osteoclast fusion-related genes, Nfatc1 and DC-STAMP. The expression of d2 isoform of vacuolar ATPase V0 domain (Atp6v0d2) and c-Fos, however, were comparable in the Control and KO mice. FIG. 4C is a Western blot showing the similar levels of c-Fos but significant decrease of Naftc1 in the above cells; Erk2 was used as a loading control. FIG. 4D is a series of images showing that over-expression of Nfatc1 in Shp2 KO bone marrow cells restores the ability of these cells to form multinucleated osteoclasts. Bone marrow cells isolated from KO mice were infected with retrovirus (see the method section for details) expressing either GFP or NFATc1/GFP. The left panel shows the infection efficiency visualized by GFP expression at day 3 post infection (blue dots are nuclei stained with DAPI). The right panel demonstrates the restored formation of multinucleated osteoclasts (arrows) from cultured bone marrow cells of Shp2 KO mice. *p<0.05 (student t-test) compared to the untreated control group.

FIGS. 5A-B are a series of images and graphs that illustrate that pharmacological inhibition of Shp2 in vitro blunts osteoclast formation and augments the effect of bisphosphonates on osteoclast-mediated bone resorption. FIG. 5A is a series of images depicting TRAP staining (left) that shows the effects of Shp2 inhibitor NSC-87877 (50 nM), Aldronates (25 nM) or a combination of NSC-87877 (50 nM) and Aldronates (25 nM) on the formation of multinucleated osteoclasts from wild type bone marrow cells in ex vivo osteoclast culture. Toluidine blue staining (right) demonstrates the pits (arrows) formed on dentin slices after being co-cultured with osteoclasts for 10 days under the same culture conditions as described in A. Culture medium was changed every 3 days. Bar=100 um. FIG. 5B is a set of bar graphs that show the average number of multinucleated osteoclasts (>3 nucleus) (top panel) and the average number of pits on dentin slices in the different culture conditions as described in A (bottom panel), n=3. *, p<0.05 (Student t-test) compared to the untreated control group. ̂p<0.05 (Student t-test) compared to single chemical treated groups.

FIG. 6A is a series of images comparing a Ctsk-expressing control mouse to a Ctsk-expressing Shp2 KO mouse to demonstrate the increase of bone mineral density in Shp2 KO mice; bottom panels depict the normal tooth eruption in both the control and Shp2 KO mice). FIG. 6B is a series of x-ray and micro-CT images comparing an OC control mouse to an OC Shp2 KO mouse at 2, 8, and 16 weeks of age, depicting bone mass in a joint region.

FIG. 7 is a multiple sequence alignment of Shp2 homologs as depicted in J. Cell Biochem., 2011, 112(8):2062-2071, incorporated herein by reference.

FIGS. 8A-B are electron microscopic images showing the size of polyethylene (A) and titanium particles used in Example 8. PeP: polyethylene particles; TiP: titanium particles. FIGS. 8C-D are phase contrast (C) and electron microscopic (D) images demonstrating the engulfment of PeP (C, dark circles) and TiP (D) particles by bone marrow derived macrophages (BMM).

FIG. 9 is a set of 5 bar graphs of qRT-PCR results showing that PeP and TiP treatment of BMM induces the expression of osteoclastogenic genes: c-Fos, Nfatc1, MMP9, Trap, and Ctsk. This differentiation process was inhibited by the inhibition of Shp2 enzymatic activity with NSC87877 (NSC). MR represents the value of positive controls (BMM cultured in osteoclast medium).

FIGS. 10A-B are a set of images and graphs showing that pharmacological inhibition of Shp2 blunted osteoclast-mediated bone resorption. FIG. 10A is a set of 4 images of pit assays demonstrating that Shp2 deletion (Ctsk-KO) and chemical inhibition with NSC87877 markedly reduced osteoclast-mediated bone resorption. Toluidine blue staining was used to visualize the pits formed on dentin slices after being co-cultured with osteoclasts for 10 days. FIG. 10B is a set of 2 bar graphs showing the quantitative data of FIG. 10A. Data was presented as the percentage of pit numbers relative to the controls. n=3. *p<0.05 (Student's t-test; compared to the control group).

### DETAILED DESCRIPTION

Osteoclasts play a key role in maintaining bone mass and sustaining healthy bone architecture.

Inhibition of osteoclast maturation has broad applications in the treatment of osteoclast-mediated skeletal disease, especially osteoporosis and peri-prosthetic osteolysis. The methods and compositions described herein reduce bone loss by blocking the fusion of preosteoclasts and reducing the number of mature osteoclasts. Manipulation of Shp2 activity modulates bone mineral loss and affects osteoclast number and activity. Elevated numbers and/or activity of osteoclasts play a crucial role in the development of osteoporosis and periprosthetic osteolysis. Substantial attempts have been dedicated during the past decades to invent medications that can block or slow down bone loss by suppressing osteoclast number and/or activity, leading to the discovery and widespread use of Bisphosphonates and their derivatives, and denosumab. As described above, these medications, such as Bisphosphonates, have disadvantages and adverse side effects.

The present invention provides superior compositions, methods, and devices for treating and/or preventing osteoporosis or a osteolytic disorder, such as peri-prosthetic osteolysis.

Osteolysis

Periprosthetic osteolysis is the most common complication after primary arthroplasty. Components used in arthroplasty generate debris, or wear particles, caused by abrasion. These wear particles lead to a localized inflammatory response, which causes the release of various cytokines that affect osteoclast differentiation and activity (e.g., TNF, RANKL, IL-6, IL-1, and IL-11). The prolonged duration of inflammatory activity promotes progressive osteolysis. Periprosthetic osteolysis is progressive and may be complicated by joint failure or periprosthetic fracture with the subsequent need for surgical revision. Thus, diagnostic imaging is helpful in accurately evaluating the extent and distribution of osteolysis. For example, radiographs are traditionally used to characterize and monitor periprosthetic osteolysis. Geographic or linear zones of periprosthetic lucency greater than 2 mm that progress on serial examinations or develop after 2 years following arthroplasty are indicative of osteolysis. Computed tomograph (CT) can sometimes provide a more sensitive detection of images characteristic of osteolysis. For example, radiolucent lesions that communicate with the joint space and have well-defined sclerotic borders- are indicative of osteolysis. Magnetic resonance imaging (MRI) is also used to diagnose periprosthetic osteolysis. MRI provides higher sensitivity than CT in detection of small (less than 3 cm) periprosthetic lesions. MRI can also be used to identify extraosseous soft tissue deposits, pathology affecting neurovascular bundles, or precursors to bone resorption. Unlike infections, osteolysis presents as well-defined lesions with low signal intensity similar to skeletal muscle by MRI. (Desai, M. A. et al. Orthopedics 2008, 31(6).)

Implants (e.g., Orthopedic/Dental)

Inhibitors of Shp2 or its signaling partners, such as Nfatc1, are applied onto or into prosthetic devices or other orthopedic/dental hardware such as screws or pins to suppress local osteoclastogenesis triggered by prosthetic wear debris. This application also includes coupling inhibitors for Shp2, Nfatc1 and its other signaling partners to prosthesis for suppressing local osteoclastogenesis.

The inhibitors are incorporated into the material from which the device or hardware is made or applied onto, e.g., dipped or sprayed, with compositions as a surface coating. For example, the inhibitors are mixed with titanium-based, glass-based, polymer-based, hydroxyapatite-based, or other calcium phosphate-based coatings to locally inhibit bone loss at the site of implantation or insertion into bone or cartilage. In another example, the inhibitors are incorporated into bone cement, which is used to secure orthopedic/dental implants.

Methods for making coatings containing bioactive compounds have been described, e.g., U.S. Pat. No. 8,075,562 (hereby incorporated by reference).

Osteoporosis

Osteoporosis is a systemic skeletal disease that is characterized by low bone density and deterioration of bone tissue, leading to an increase in fragile bones and susceptibility to fracture. Osteoporosis manifests as a fracture of a vertebra or backbone, hip, forearm, or any bony site where sufficient bone mass is lost. These fractures often occur after apparently mild stress, such as bending over, falling, lifting, or jumping from a standing position. In later stages of the disease, disfigurement, pain, and debilitation commonly occur. Subjects at risk for osteoporosis include subjects with risk factors, such as women age 65 or older (e.g., age 65, 70, 75, 80, 85, 90, or older), men age 70 or older (e.g., age 70, 75, 80, 85, 90, or older), postmenopausal women age 65 or younger (e.g., age 65, 60, 55, 45, 40, 35, 30, or younger) with one or more risk factors for osteoporosis, men ages 50-70 (e.g., age 50, 55, 60, 65, or 70) with one or more risk factors for osteoporosis, and men or women age 50 or older (e.g., age 50, 55, 60, 65, 70, 75, 80, 85, 90, or older) who have suffered a fracture. Risk factors for osteoporosis include long-term use of medications associated with low bone mass or bone loss (e.g., corticosteroids, anti-seizure medications, Depo-Provera, thyroid hormone, and aromatase inhibitors), especially long-term use of corticosteroids (i.e., more than 5 mg/day for more than 3 months), a history of prostate cancer or breast cancer treatment, a history of diabetes, reduced calcium absorption in the gut, prior gastric surgery, caffeine intake, cigarette smoking, family history of fracture or fragility, physical inactivity, estrogen or thiazide use, alcohol consumption, calcium, vitamin K, or vitamin D deficiency, thyroid imbalances, estrogen or testosterone deficiencies, early menopause, anorexia nervosa, rheumatoid arthritis, significant loss of height, and significant weight loss or low body mass index. (Prevention and Management of Osteoporosis. WHO Technical Report Series, 921. 2003.)

Diagnosis of osteoporosis is made by using a number of techniques to measure bone mass or bone density, including but not limited to single X-ray absorptiometry (SXA), dual-energy x-ray absorptiometry (DXA), ultrasound, quantitative computed tomography (QCT) scan, radiography, and MRI. Bone density measurements are generally provided as Z and T scores compared to the average bone density of the young healthy population. A T score provides the standard deviation of a subject relative to the normal value in young adults. A Z score provides the standard deviation of a subject relative to the normal value in the subject's own body size and age group. A T score greater than or equal to −1 (i.e., a value of bone mineral density within 1 standard deviation of the adult reference mean) indicates normal bone density. A T score between −2.5 and −1 (i.e., a value of bone mineral density more than 1 standard deviation below but less than 2 standard deviations below the young adult mean) indicates low bone mass, or osteopenia. A T-score lower than or equal to −2.5 (i.e., a value of bone mineral density 2.5 standard deviations or more below the young adult mean) indicates osteoporosis. A bone mineral density 2.5 or more standard deviations below the young adult mean in the presence of one or more fragility fractures indicates severe osteoporosis. Osteopenia is generally considered a precursor to osteoporosis. (Prevention and Management of Osteoporosis. WHO Technical Report Series, 921. 2003.)

An effective therapeutic amount of a compound (e.g., a Shp2 pathway inhibitor) for the treatment or prevention of osteoporosis is about 0.1 mg/kg body weight to about 1000 mg/kg body weight per day. Exemplary dosages are about 0.1 mg/kg to about 500 mg/kg per day (e.g., about 0.1 mg/kg to about 50 mg/kg per day, or about 0.1 mg/kg to about 5 mg/kg per day). Effective doses will also vary, as recognized by those skilled in the art, depending on the severity of osteoporosis being treated, route of administration, excipient usage, and the possibility of co-usage with other therapeutic treatments (e.g., bisphosphonate compound).

Bone Formation and Remodeling

Two types of cells regulate bone formation and remodeling: osteoblasts and osteoclasts. Osteoblasts make new bone, and osteoclasts resorb bone. The present invention relates to osteoclasts.

Osteoclasts (OC), the exclusive bone resorbing cells in mammals, play an essential role in skeletal development and remodeling, including maintaining bone mass and sustaining healthy bone architecture. OC origins from hematopoietic cells; their differentiation along OC lineage requires two key factors: macrophage colony stimulating factor (M-CSF) and receptor activator of nuclear factor-κB ligand (RANKL) (Teitelbaum S L, et al. (2003) Nat Rev Genet 4(8):638-49; Asagiri M, et al. (2007) Bone 40(2):251-64; Boyle W J, et al. (2003) Nature 423(6937):337-42). M-CSF signals through its receptor c-Fms, a transmemberan receptor tyrosine kinase that is crucial upon activation for the proliferation and survival of myeloid cells and osteoclast precursors (OCP)s (Pixley F J, et al. (2004) Trends Cell Biol 14(11):628-38; Ross FP (2006) Ann N Y Acad Sci 1068:110-6). RANKL is a member of TNF superfamily cytokines that was originally identified as an activator for dendritic cells (Anderson D M, et al. (1997) Nature 390(6656):175-9); it turns out that RANKL, besides its function in immune system (Leibbrandt A, et al. (2008) Ann N Y Acad Sci 1143:123-50) and mammary gland development (Fata J E, et al. (2000) Cell 103(1):41-50), plays a pivotal role in osteoclastogenesis. Upon RANKL binding of RANK, RANKL elicits a broad range of signaling responses essential for OCP to differentiate into OC and also for OC bone resorptive activity (Wada T, et al. (2006) Trends Mol Med 12(1):17-25; Luo L Q, et al. (2008) Guang Pu Xue Yu Guang Pu Fen Xi 28(3):704-6). The importance of M-CSF/c-Fms and RANKL/RANK signaling in osteoclastogenesis and OC function regulation has been documented by the osteopetrotic phenotype of mice missing a gene for any of these proteins (Kong Y, et al. (1999) Nature 397(6717): p 315-23; Dougall W C, et al. (1999) Genes Dev 13(18):2412-24; Yoshida H, et al. (1990) Nature 345(6274):442-4).

At the molecular level, M-CSF binding of c-Fms can induce receptor dimerization and activate c-Fms kinase activity. Consequently this activation leads to autophosphosprylation of c-Fms on selected tyrosine residues; these residues then recruit intracellular signaling relaying molecules that contain either Src homology 2 or phosphotryosine binding domains, such as Src family kinase members (SFKs) (Alonso G, et al. (1995) J Biol Chem 270(17):9840-8), Grb2 and p85 subunit of PI3Kinase etc. (Pixley F J, et al. (2004) Trends Cell Biol 14(11):628-38; Ross FP (2006) Ann N Y Acad Sci 1068:110-6; van der Geer P, et al. (1993) Embo J 12(13):5161-72). At last, activation of c-Fms can transduce signals along Ras/Erk, phosphatidylinositide 3-kinase (PI3 kinase)/Akt (also known as protein kinase B), Signal Transducer and Activator of Transcription (stat), c-Casitas B-lineage Lymphoma (c-cbl), and phospholipase C-gamma (PLCγ), etc. signaling cascades to modulate various biological responses. Activation of Ras/Erk, PI3kinase/Akt, and PLCγ pathways primarily mediates signals for the proliferation and survival of myeloid cells and OCP (Roth P, et al. (1992) Curr Top Microbiol Immunol 181:141-67; Tanaka S, et al. (1993) J Clin Invest 91(1):257-63). In contrast, activation of RANK upon RANKL binding in OCP and OC primarily promotes multinucleated OC formation and function (Teitelbaum S L, et al. (2003) Nat Rev Genet 4(8):638-49; Wada T, et al. (2006) Trends Mol Med 12(1):17-25); transgenic mice deficient RANKL (Kong Y Y, et al. (1999) Nature 397(6717):315-23) or RANK (Dougall W C, et al. (1999) Genes Dev 13(18):2412-24) completely lack osteoclasts and develop severe osteopetrosis.

The signaling mechanism for RANKL in osteoclasts has been investigated. Binding of RANKL to its receptor RANK promotes receptor trimerization and its recruitment of members of TNF receptor-associated factors (TRAF) and Gab family adaptor proteins (Wada T, et al. (2005) Nat Med 11(4):394-9; Taguchi Y, et al. (2009) Genes to Cells 14(11):1331-1345); these interactions are required for the subsequent activation of nuclear factor kappa-light-chain-enhancer of activated B cells (NF-kB) and c-Jun N-terminal kinase (JNK) pathways and expression of c-Fos (Grigoriadis A E, et al. (1994) Science 266(5184):443-8) and nuclear factor of activated T cells (NFATc1) (Takayanagi H, et al. (2002) Dev Cell 3(6):889-901), a calcineurin- and calcium-regulated transcription factor reported as a key factor to regulate osteoclastogenesis. NFATc1−/− embryonic stem (ES) cells are defective to differentiate into osteoclasts and overexpression of NFATc1 can circumvent the requirement of RANKL to induce ES cell differentiation into osteoclasts (Takayanagi H, et al. (2002) Dev Cell 3(6):889-901). Mechanistically, NFATc1 can induce the expression of osteoclastogenic genes, such as tartrate resistant acid phosphatase (TRAP), cathepsin K (Ctsk), the d2 isoform of vacuolar ATPase V0 domain (ATP6v0d2) and dentritic cell-specific transmembrane protein (DC-STAMP), calcitonin receptor and matrix metal protein 9 (MMP9), by binding to the NFAT-binding sites in the promoter regions of these genes. Therefore, NFATc1 is a master regulator of RANKL-evoked osteoclast differentiation, fusion, and function activation. In addition, negative regulator of RANKL signaling has also been reported. RANKL have a decoy receptor, osteoprotegerin (OPG), which inhibits RANKL action by competing with RANK for binding RANKL (Simonet W S, et al. (1997) Cell 89(2):309-19; Bucay N, et al. (1998) Genes Dev 12(9):1260-8). At the molecular level, how these signaling pathways are regulated and the genes that regulate osteoclast development and function under physiological and disease conditions, however, remained incompletely understood prior to the present invention.

Shp2

Shp2, encoded by the gene, tyrosine-protein phosphatase non-receptor type 11 (PTPN11), is a ubiquitously expressed cytoplasmic, non-receptor, src-homology-2 domain containing, protein tyrosine phosphatase. Shp2 contains two tandem Src homology-2 (SH2) domains. It has been implicated as a critical component downstream of M-CSF receptor and RANK to modulate osteoclast development and function. It is required for Ras/Erk activation by most, if not all, receptor tyrosine kinases (e.g., M-CSF receptor c-Fms) in cytokine receptor signaling and influences various biological aspects of cells including viability, proliferation, migration, and differentiation (Neel B G, et al. (2009) Handbook of Cell Signaling (2nd edition):771-809; Grossmann K S, et al. (2010) Adv Cancer Res 106:53-89). Postnatal Shp2 deficiency in various tissues/cells has diverse effects on their development and function. Previous studies of tissue/cell-specific Shp2 deficient mice uncovered an important role for Shp2 in muscle, central nervous system, liver, T-cell and hematopoetic stem cells; both a positive and negative role for Shp2 has downstream multiple receptor tyrosine kinase and cytokine receptors to influence cell viability, proliferation and differentiation. However, prior to this invention, little was known about how Shp2 functions in osteoclasts and skeletal remodeling (i.e., in osteoclastogenesis and bone homeostasis in vivo) due to early embryonic lethality caused by global Shp2 deficiency.

Previous studies using primary cells and cell lines described that Shp2 is critical for c-Fms (Lee A W, et al. (2006) Cell Death Differ 13(11):1900-14) and RANK signaling (Wada T, et al. (2005) Nat Med 11(4):394-9), suggesting a role for Shp2 in regulating osteoclastogenesis, skeletal remodeling, and bone mineral homeostasis. Indeed, mice with ubiquitous Shp2 deletion postnatal have skeletal malformation and bone marrow cells from these Shp2 mutant mice failed to make TRAP+ multinucleated osteoclasts (Bauler T J, et al. (2011) Disease Models & Mechanisms 4(2):228-239). However, prior to the present invention, little was known about the how M-CSF and RANK-evoked signaling, specifically signal induces terminal differentiation of OC, are regulated by Shp2 in vivo since Shp2 protein null mutation in mice causes early embryonic lethality (Yang W, et al. (2006) Dev Cell 10(3):317-27; Saxton T, et al. (1997) EMBO J 16(9): p2352-64).

As described in detail below and in the Examples, in the present invention, a Shp2 expression was specifically ablated in Ctsk-expressing cells, likely osteoclasts, via a “Cre-loxP”-mediated gene deletion. Mice lacking Shp2 in OC were retarded in growth and osteopetrotic, manifesting as increased bone mineral density, increased bone volume over total volume (BV/TV), increased trabecular bone number but decreased trabecular thickness and spacing. Shp2 deficiency in Ctsk-expressing cells inhibited the formation of TRAP+ multinucleated osteoclasts in vivo and ex vitro upon M-CSF and RANKL induction. In vitro osteoclastogenesis assays revealed that this defect is caused by the blockade of pre-osteoclast fusion rather than their viability or proliferation. This observation was further confirmed by the gene expression analysis described in greater detail in the Examples, which showed that a panel of osteoclastogenic marker genes was downregulated in the absence of Shp2. Mechanistically, Shp2 was required for RANKL-evoked NFATc1 expression; overexpression of NFATc1 in Shp2 deficient bone marrow cells rescued preosteoclast fusion and multiosteoclast formation, and chemical inhibition of Shp2 activity ex vivo recapitulated the effect of Shp2 deletion on osteoclastogenesis in vivo.

Shp2 Inhibitors

Shp2 or its regulated pathway are manipulated to treat osteoporosis and osteolytic diseases. Shp2 inhibitors reduce osteoclastogenesis and thus the formation of mature osteoclasts, which in turn leads to a decrease in bone resorption and clinical benefit for patients suffering from those disorders.

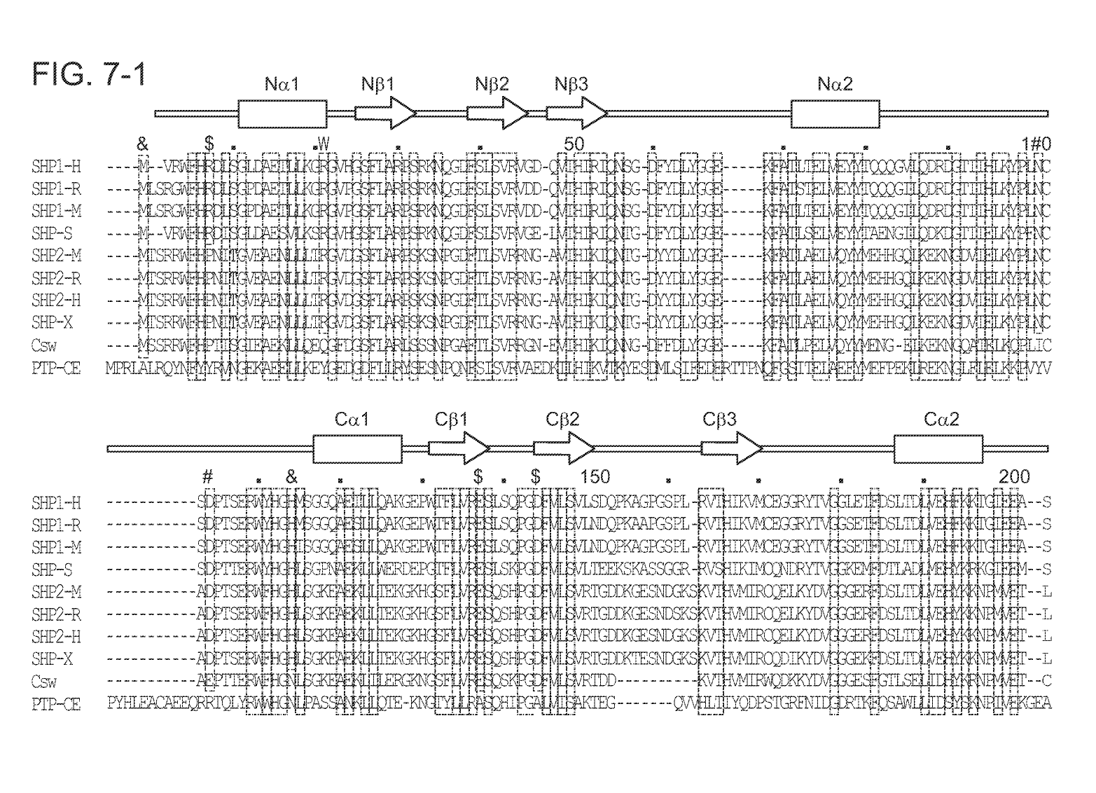

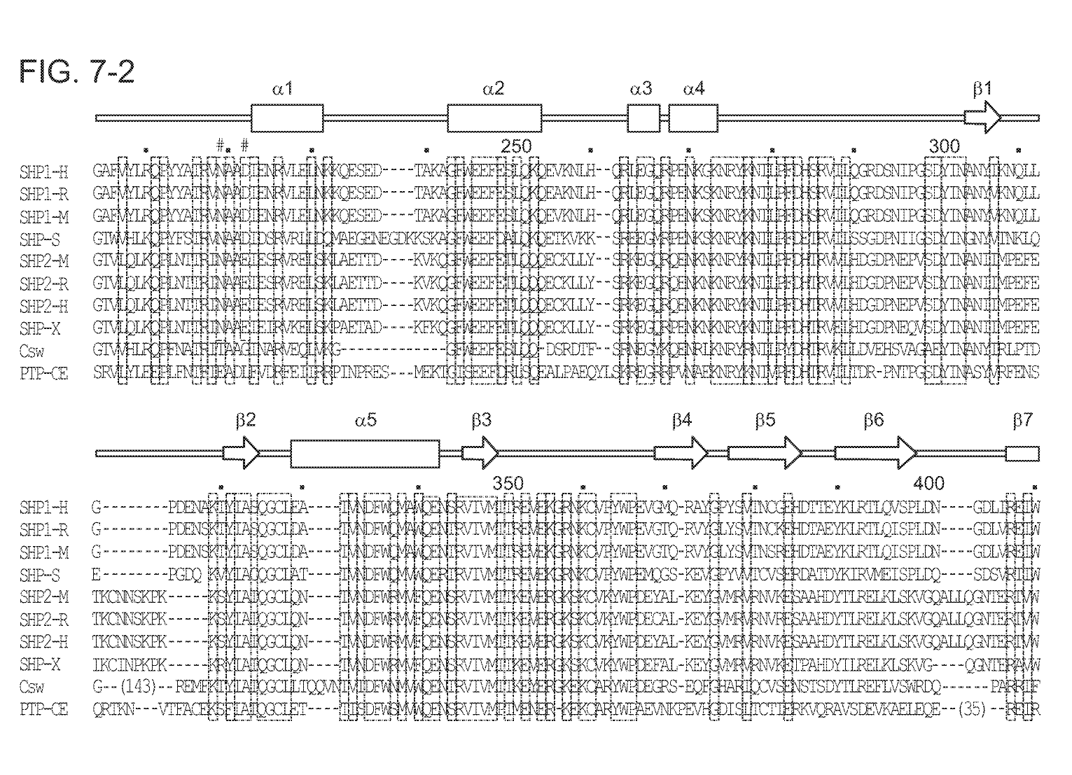

A number of Shp2 inhibitors have been described. For example, the Shp2 inhibitor binds to the Shp2 catalytic site. An Shp2 inhibitor that binds to the PTP domain of Shp2 is described in Bentires-Alj, M. et al. Cancer Res. 2004, 64:8816-8820. In another example, the Shp2 inhibitor binds to one or more amino acids in human Shp2, where the one or more amino acids are selected from the group consisting of K280, Y279, N280, R362, K364, K366, W423, P424, D425, H426, G427, S460, A461, I463, G464, R465, and Q510, or homologous residues thereof in non-human Shp2 (Chen, L. et al. Mol. Pharmacol., 2006, 70:562-570; Chen, L. et al., Biochem. Pharmacol., 2010, 15; 80(6):801-10; Hellmuth, K. et al. Proc. Natl. Acad. Sci., 2008, 105:7275-7280; and Zhang, X. et al. J. Med Chem. 2010, 53(6):2482-2493). A homologous residue in a non-human Shp2 is a residue that aligns in a multiple sequence alignment with a corresponding residue in human Shp2 (FIG. 7) (J. Cell. Biochem., 2011, 112(8):2062-2071, incorporated herein by reference). In yet another example, the Shp2 inhibitor prevents binding of Shp2 to a binding partner, e.g., Gab 1 or Gab2.

An exemplary small molecule Shp2 inhibitor is NSC 87877 (Chen, L. et al. Mol. Pharmacol., 2006, 70:562-570). Others include SPI-112 (Chen, L. et al., Biochem. Pharmacol., 2010, 15; 80(6):801-10), SPI-112Me (Chen, L. et al. Biochem. Pharmacol., 2010, 15; 80(6):801-10), PHPS1 (Hellmuth, K. et al. Proc. Natl. Acad. Sci., 2008, 105:7275-7280), SHP2 inhibitor II-B08 (3-(1-(3-(Biphenyl-4-ylamino)-3-oxopropyl)-1H-1,2,3-triazol-4-yl)-6-hydroxy-1-methyl-2-phenyl-1H-indole-5-carboxylic acid) (Zhang, X. et al. J. Med Chem. 2010, 53(6):2482-2493), C21 (Yu, Z., et al. Bioorganic and Medicinal Chemistry Letters 2011; 21(14):4238-4242.), tautomycetin (TTN) (Liu, S., et al. 2011. Chem. Biol. 18, 101-110), TTN D-1 (Liu, S., et al. 2011. Chem. Biol. 18, 101-110), 7-deshydroxypyrogallin-4-carboxylic acid (DCA) (Wu, D., et al. (2009) PLoS ONE 4(3): e4914), NSC-117199 (Lawrence, H. R., et al. J. Med. Chem., 2008, 51 (16):4948-4956), 8Z,11Z-Feptadecadienoic acid (Liu, D. et al. Bioorg Med Chem Lett. 2011 Nov. 15; 21(22):6833-7), 14Z,17Z-tricosadienoic acid (Liu, D. et al. Bioorg Med Chem Lett. 2011 Nov. 15; 21(22):6833-7), caffeic acid (Liu, D. et al. Bioorg Med Chem Lett. 2011 Nov. 15; 21(22):6833-7), 2-hydroxy-3-[(1-oxododecyl)oxy]propyl-β-d-glucopyranoside (Liu, D. et al. Bioorg Med Chem Lett. 2011 Nov. 15; 21(22):6833-7), and derivatives of the Shp2 inhibitors described herein. Other exemplary Shp2 pathway inhibitors include one or more of the compounds described in U.S. 2008/0176309, incorporated herein by reference.

The chemical structures of some of these inhibitors are shown below.

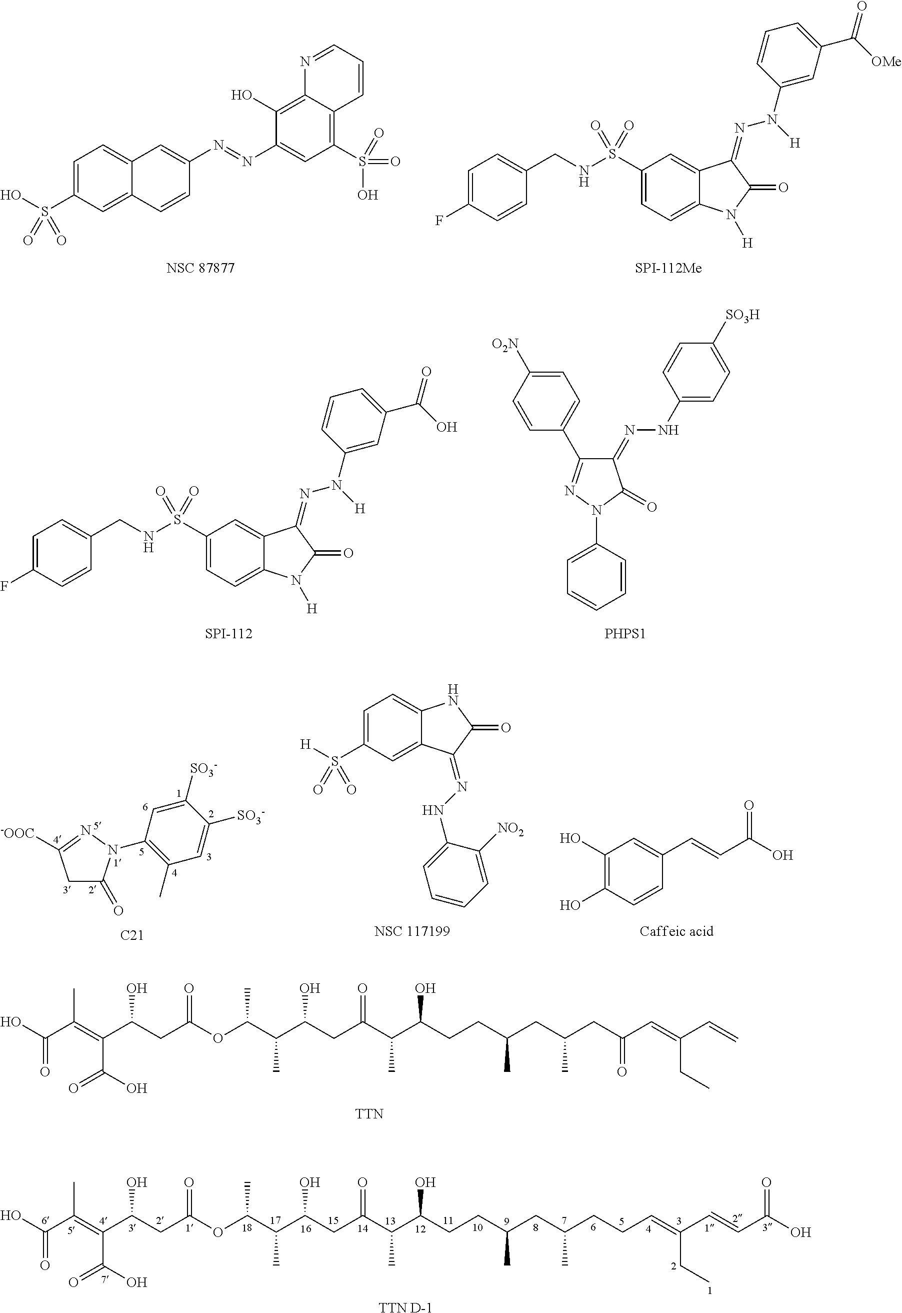

NSC 87877 is suitable for administration to humans. The compound has been delivered to a mouse model of prostate cancer in the form of loaded nanoparticles conjugated to T cells and was effective in reducing tumor burden without causing greater death than in control-treated mice. Additionally, mice given NSC-87877 did not die significantly sooner than control-treated or untreated mice. These results are evidence of lack of toxicity of NSC 87877 in vivo. (Stephan, M. T., et al. Biomaterials, 2012, 33:5776-5787).

Other Shp2 pathway inhibitors include those that inhibit the following proteins: Gab2, Nfatc1, Calcitonin receptor, Ctsk, DC-STAMP, Mmp9, and Trap.

Shp2 Regulation of Preosteoclast Fusion

M-CSF is required for the proliferation and survival of osteoclast precursors/macrophages, and their expression of RANK. M-CSF signals through c-Fms, which is a receptor tyrosine kinase. Shp2 was found in the c-Fms-associated signaling complex upon M-CSF binding, suggesting a role for Shp2 in regulating the proliferation and survival of osteoclast precursors/BMM and osteoclasts. In the present invention, Shp2 expression was specifically ablated in osteoclasts, specifically, Ctsk-expressing osteoclast lineage cells. Surprisingly, it was found that Shp2 deletion has minimal effect on the growth and survival of osteoclast precursors and pre-osteoclasts in the presence of M-CSF and RANKL induction as determined by WST-1 assays. This observation is consistent with data showing that deletion of Shp2 in bone marrow derived macrophages (via LysM-Cre) had only a mild effect on M-CSF-evoked cell proliferation, viability, and differentiation (Yang, W., data not presented). Surprisingly, Shp2 regulates osteoclastogenesis by promoting osteoclast fusion rather than supporting growth and survival of osteoclast precursors and pre-osteoclasts. Mice lacking Shp2 in macrophages (mediated by lysMCre) have normal circulating blood cell counting and also differential counts. While the numbers of TRAP expressing preosteoclasts in the cultures were comparable between Control and KO mice, the deletion of Shp2 severely impairs the formation of multinucleated osteoclasts both in vivo and ex vivo. Moreover, Shp2 deficiency in these cells led to elevated tyrosyl phosphorylation of cellular proteins and altered Erk and Akt activation upon RANKL stimulation. M-CSF signals through its receptor c-Fms, which is a receptor tyrosine kinase. Activation of c-Fms upon M-CSF binding can trigger intracellular signaling along pathways to support cell proliferation and was reported to require Shp2. It has been demonstrated that inactivated Shp2 expression in OCP/bone; it transduces signals to M-CSF signals through its receptor c-Fms and activates multiple intracellular signaling pathways including Ras/Erk, PI3 Kinase/Akt, Stats, and PLCγ. Activation of one or more of these pathways supports the proliferation and myelomonocytic differentiation of bone marrow (BM) cells. Shp2 is required for M-CSF-evoked Erk activation but negatively regulates PI3 kinase/Akt signaling cascade (Yang, W., data not presented). In monocytes/macrophages, Shp2 associates with Gab2 in a signaling protein complex upon M-CSF stimulation. Yet, its precise roles in M-CSF signaling, as well as its effects on monocytes/macrophages and osteoclastogenesis in vivo, were not understood prior to the present invention. Taken together, the results presented herein demonstrate that Shp2 primarily regulates osteoclastogenesis by influencing the fusion of preosteoclasts.

Shp2 Effect on Nfatc1 Expression

As described above, Shp2 deficiency has minimal impact on proliferation and viability of pre-osteoclasts but dramatic interference on their fusion. This observation is further supported by the decreased expression of a panel of osteoclastogenic genes (Calcitonin receptor, Ctsk, DC-STAMP, Mmp9, Trap, and Nfatc1) in pre-osteoclasts with Shp2 deficiency. Previously published gene expression data have identified these genes as the primary effectors upon RANKL stimulation. Among them, Nfatc1 and its downstream effector, DC-STAMP, were reported previously to be critical for osteoclast fusion. Importantly, overexpression of Nfatc1 in BM cells lacking Shp2 restores their ability to make giant multinucleated osteoclasts, supporting a model that Shp2 is a signaling component of the RANK pathway in regulating osteoclast terminal differentiation and it is essential for Nfatc1 expression and preosteoclast fusion.

Previous studies have shown that c-Fos is upstream of Nfatc1 and that both are required for pre-osteoclast fusion in response to RANKL signaling. Moreover, Nfatc1 directly controls ATP6v0d2 and DC-STAMP gene expression. The results presented herein and described in greater detail in the Examples are surprising in that in Shp2 KO osteoclasts, only Nfatc1 and DC-STAMP levels were significantly reduced while c-Fos and Atp6v0d2 expression levels remained unchanged in response to both M-CSF and RANKL stimulation. This indicates that Shp2 is not required for transcription regulation of Atp6v0d2 and c-Fos in response to these cytokine signals.

Cooperative Effect of Shp2 Inhibition with Bisphosphonates (BPs)

Healthy bone homeostasis requires balanced activities from osteoclasts and osteoblasts. The sole inhibition of bone resorption by osteoclasts can protect existing bone from being absorbed, but does not facilitate the formation of new bone, repair microcracks in bones, or remodel bones in response to physiological changes. Osteoclast inhibitors, specifically, BPs, are widely used to treat osteoporosis. These drugs are rapidly absorbed onto the bone surface after administration and are ingested by the osteoclasts during bone resorption. Once ingested, the BPs kill the osteoclasts. Mechanistically, BPs inhibit bone resorption ability of osteoclasts by coating all bone surfaces, causing the death of activated osteoclasts, and blindly shutting down all normal and needed bone resorption that is critical for bone remodeling and repair. As a result, BPs clinical treatments have been reported to cause atypical bone fractures presumably due to lack of normal bone remodeling and repair. An ideal osteoporosis medicine should reduce the number of healthy and functional osteoclasts yet not eliminate and/or inhibit the function of entire population of osteoclasts.

As described in greater detail in the Examples, analysis of osteoclast development in Shp2 KO mice suggests a new way to control osteoclast development and possible bone dynamics. It was determined that the addition of one of the Shp2 inhibitors can reduce the dose of BPs needed to slow down bone absorption. The combined treatment of both BP and the Shp2 inhibitor at their suboptimal doses reduces osteoclast survival and bone absorption activity to the same extent as the individual chemicals can achieve at their optimal levels. An application of these findings is the development of a novel therapy for treating osteoporosis and other bone degeneration diseases. The synergistic actions of combining BP treatment with Shp2 inhibition in suppressing osteoclast formation and bone loss reduces the side effects of BP and improves current treatments of OP and other bone degeneration and osteolytic diseases.

As described above and in greater detail in the Examples, in order to understand how Shp2 functions in osteoclastogenesis and skeletal homeostasis, a tissue/cell-specific gene knockout approach was used, involving conditionally inactivated Shp2 expression in cathepsin K (Ctsk)-expressing osteoclast lineage. By analyzing osteoclast-specific Shp2 deficient mice and their cellular derivatives, it was discovered that Shp2 regulates osteoclast development and their bone resorptive activity and that Shp2 is essential for osteoclastogenesis both in vivo and ex vivo. Specifically, Shp2 deficiency in cathepsin K-expressing osteoclastogenic cells causes severe osteopetrosis and impairs the formation of functional multinucleated osteoclasts in vivo and ex vivo upon M-CSF and RANKL induction. Shp2 regulates osteoclastogenesis mechanistically by promoting the fusion of preosteoclasts rather than influencing their viability and proliferation. Indeed, gene and protein expression analyses demonstrate that Shp2 is required for RANK-induced multinucleated osteoclast formation by promoting the expression of Nfatc, a master transcription factor that is indispensable for osteoclast terminal differentiation. Shp2 appears to relay RANKL signals to increase the expression of Nfatc1, a master transcription factor that governs the fusion of pre-osteoclasts. In contrast, Shp2 deficiency has minimal effect on the M-CSF-dependent survival and proliferation of osteoclast precursors. Surprisingly, pharmacological or chemical inhibition of Shp2 activity in vitro (e.g., in osteoclast precursors) attenuates multinucleated osteoclast formation (i.e., preventing osteoclast precursors from fusing and forming mature osteoclasts) and reduces their bone resorptive ability. Also, pharmacological or chemical inhibition ex vivo recapitulates the effect of Shp2 deletion on osteoclastogenesis in vivo. It also augments the inhibition of BPs on osteoclast-mediated bone resorption as evidenced by the reduced number of pits in a Pit assay. Collectively, the findings presented herein show that the pharmacological manipulation of Shp2 or its regulated signaling pathway(s), systemically or locally (e.g., in mature osteoclasts), reduces osteoclast-mediated bone resorption and can be used for developing better treatments for skeletal disorders that result from elevated number and/or activity of osteoclasts, such as OP, peri-prosthetic osteolysis, and other osteolytic diseases.

The data described below was generated using the following materials and methods.

GenBank Accession Numbers

An exemplary amino acid sequence of murine Shp2 is provided in GenBank Accession No. NP_{—}035332.1 (GI:6755228), incorporated herein by reference. Exemplary nucleic acid sequences encoding murine Shp2 are provided in GenBank Accession No. NM_{—}011202.3 (GI:158508547) and GenBank Accession No. NM_{—}001109992.1 (GI:158508567), both of which are incorporated herein by reference.

An exemplary amino acid sequence of human Shp2 is provided in GenBank Accession No. NP_{—}002825.3 (GI: 33356177) (SEQ ID NO: 2), incorporated herein by reference. Exemplary nucleic acid sequences encoding human Shp2 are provided in GenBank Accession No. NM_{—}002834.3 (GI:33356176) and GenBank Accession No. NM_{—}080601.1 (GI:18375643), both of which are incorporated herein by reference.

An exemplary amino acid sequence of human Gab1 is provided in Genbank Accession no. NP_{—}997006.1, incorporated herein by reference. An exemplary nucleic acid sequence encoding human Gab1 is provided by Genbank Accession No. NM_{—}207123.2, incorporated herein by reference.

An exemplary amino acid sequence of human Gab2 is provided in Genbank Accession no. Q9UQC2.1, incorporated herein by reference. An exemplary nucleic acid sequence encoding human Gab2 is provided by Genbank Accession No. AB018413.1, incorporated herein by reference.

An exemplary amino acid sequence of human Nfatc1 is provided in Genbank Accession no. NP_{—}765978.1, incorporated herein by reference. An exemplary nucleic acid sequence encoding human Nfatc1 is provided by Genbank Accession No. NM_{—}172390.2, incorporated herein by reference.

An exemplary amino acid sequence of human calcitonin receptor is provided in Genbank Accession no. AAA35640.1, incorporated herein by reference. An exemplary nucleic acid sequence encoding human calcitonin receptor is provided by Genbank Accession No. L00587.1, incorporated herein by reference.

An exemplary amino acid sequence of human Ctsk is provided in Genbank Accession no. P43235.1, incorporated herein by reference. An exemplary nucleic acid sequence encoding human Ctsk is provided by Genbank Accession No. NM_{—}000396.3, incorporated herein by reference.

An exemplary amino acid sequence of human DC-STAMP is provided in Genbank Accession no. Q9H295.1, incorporated herein by reference. An exemplary nucleic acid sequence encoding human DC-STAMP is provided by Genbank Accession No. BC064844.1, incorporated herein by reference.

An exemplary amino acid sequence of human MMP9 is provided in Genbank Accession no. NP_{—}004985.2, incorporated herein by reference. An exemplary nucleic acid sequence encoding human MMP9 is provided by Genbank Accession No. NM_{—}004994.2, incorporated herein by reference.

An exemplary amino acid sequence of human Trap is provided in Genbank Accession no. P29965.1, incorporated herein by reference. An exemplary nucleic acid sequence encoding human Trap is provided by Genbank Accession No. BC111014.1, incorporated herein by reference.

An exemplary amino acid sequence of human Nfatc2 is provided in Genbank Accession no. NP_{—}036472.2, incorporated herein by reference. An exemplary nucleic acid sequence encoding human Nfatc2 is provided by Genbank Accession No. NM_{—}012340.4, incorporated herein by reference.

An exemplary amino acid sequence of human Atp6v0d2 is provided in Genbank Accession no. Q8N8Y2.1, incorporated herein by reference. An exemplary nucleic acid sequence encoding human Nfatc2 is provided by Genbank Accession No. NM_{—}152565.1, incorporated herein by reference.

An exemplary amino acid sequence of human c-Fos is provided in Genbank Accession no. CAA24756.1, incorporated herein by reference. An exemplary nucleic acid sequence encoding human Nfatc2 is provided by Genbank Accession No. NM_{—}005252.3, incorporated herein by reference.

An exemplary murine Shp2 protein sequence (Genbank accession No. NP_{—}035332) is shown below.

```
(SEQ ID NO: 1)
    1 mtsrrwfhpn itgveaenll ltrgvdgsfl arpsksnpgd ftlsvrrnga vthikiqntg
  61 dyydlyggek fatlaelvqy ymehhgqlke kngdvielky pincadptse rwfhghlsgk
121 eaeklltekg khgsflvres qshpgdfvls vrtgddkges ndgkskvthv mircqelkyd
181 vgggerfdsl tdlvehykkn pmvetlgtvl qlkqplnttr inaaeiesrv relsklaett
241 dkvkqgfwee fetlqqqeck llysrkegqr qenknknryk nilpfdhtrv vlhdgdpnep
301 vsdyinanii mpefetkcnn skpkksyiat qgclqntvnd fwrmvfqens rvivmttkev
361 ergkskcvky wpdeyalkey gvmrvrnvke saandytlre lklskvgqal lqgntertvw
421 qyhfrtwpdh gvpsdpggvl dfleevhhkq esivdagpvv vhcsagigrt gtfividili
481 diirekgvdc didvpktiqm vrsqrsgmvq teaqyrfiym avqhyietlq rrieeeqksk
541 rkgheytnik yslvdqtsgd qsplppctpt ppcaemreds arvyenvglm qqqrsfr
```

An exemplary human Shp2 (Tyrosine-protein phosphatase non-receptor type 11 isoform 1) 593 amino acid protein sequence (Genbank Accession No. NP_{—}002825.3) (SEQ ID NO: 2) is shown below.

```
(SEQ ID NO: 2)
    1 mtsrrwfhpn itgveaenll ltrgvdgsfl arpsksnpgd ftlsvrrnga vthikiqntg
  61 dyydlyggek fatlaelvqy ymehhgqlke kngdvielky plncadptse rwfhghlsgk
121 eaeklltekg khgsflvres qshpgdfvls vrtgddkges ndgkskvthv mircqelkyd
181 vgggerfdsl tdlvehykkn pmvetlgtvl qlkqplnttr inaaeiesrv relsklaett
241 dkvkqgfwee fetlqqqeck llysrkegqr qenknknryk nilpfdhtrv vlhdgdpnep
301 vsdyinanii mpefetkcnn skpkksyiat qgclqntvnd fwrmvfgens rvivmttkev
361 ergkskcvky wpdeyalkey gvmrvrnvke saahdytlre lklskvgqgn tertvwqyhf
421 rtwpdhgvps dpggvldfle evhhkqesim dagpvvvhcs agigrtgtfi vidilidiir
481 ekgvdcdidv pktiqmvrsq rsgmvqteaq yrfiymavqh yietlqrrie eeqkskrkgh
541 eytnikysla dqtsgdqspl ppctptppca emredsarvy envglmqqqk sfr
```

Residues 5-103 of human Shp2 (SEQ ID NO: 3, shown in underlined text in SEQ ID NO: 2) comprise the N-terminal Src homology 2 (N—SH2) domain of Shp2. Residues 13, 32, 53, and 55 of human Shp2 form a phosphotyrosine binding pocket of Shp. Residues 205-593 (SEQ ID NO: 4) of human Shp2 form the PTP domain, shown in italics in SEQ ID NO: 2 above. The amino acid sequence, residues 457-467 of human Shp2, vhcsagigrtg (SEQ ID NO: 5), forms a protein tyrosine phosphatase motif, which is part of the PTP domain of Shp2. Amino acid residues in human Shp2 that are important for its catalytic activity include K280, Y279, N280, R362, K364, K366, W423, P424, D425, H426, G427, S460, A461, I463, G464, R465, and Q510. In some cases, the catalytic site of human Shp2 is made up of one or more of K280, Y279, N280, R362, K364, K366, W423, P424, D425, H426, G427, S460, A461, I463, G464, R465, and Q510.

A fragment of the Shp2 protein contains a portion of SEQ ID NO: 2 and contains less than 593 or fewer, 550 or fewer, 500 or fewer, 450 or fewer, 400 or fewer, 350 or fewer, 300 or fewer, 250 or fewer, 200 or fewer, 150 or fewer, 100 or fewer, 80 or fewer, 70 or fewer, 60 or fewer, 50 or fewer, 40 or fewer, 30 or fewer, 20 or fewer, or 10 or fewer amino acids. In some embodiments, a fragment of the Shp2 protein contains the protein tyrosine phosphatase domain of Shp2 or a fragment thereof (e.g., having 388 or fewer, 350 or fewer, 300 or fewer, 250 or fewer, 200 or fewer, 150 or fewer, 100 or fewer, 80 or fewer, 70 or fewer, 60 or fewer, 50 or fewer, 40 or fewer, 30 or fewer, 20 or fewer, or 10 or fewer amino acids). In other embodiments, a fragment of the Shp2 protein contains the N—SH2 domain of Shp2 or a fragment thereof (e.g., having 98 or fewer, 80 or fewer, 70 or fewer, 60 or fewer, 50 or fewer, 40 or fewer, 30 or fewer, 20 or fewer, or 10 or fewer amino acids).

A fragment of a nucleic acid encoding the Shp2 protein contains a portion of the nucleic acid sequence set forth in GenBank Accession No. NM_{—}002834.3 and contains 6300 or fewer, 6000 or fewer, 5500 or fewer, 5000 or fewer, 4000 or fewer, 3000 or fewer, 2000 or fewer, 1900 or fewer, 1800 or fewer, 1700 or fewer, 1600 or fewer, 1500 or fewer, 1400 or fewer, 1200 or fewer, 1000 or fewer, 900 or fewer, 800 or fewer, 700 or fewer, 600 or fewer, 500 or fewer, 400 or fewer, 300 or fewer, 200 or fewer, 100 or fewer, 90 or fewer, 80 or fewer, 70 or fewer, 60 or fewer, 50 or fewer, 40 or fewer, 30 or fewer, 20 or fewer, 10 or fewer nucleotides.

In some cases, a compound (e.g., small molecule) or macromolecule (e.g., nucleic acid, polypeptide, or protein) of the invention is purified and/or isolated. As used herein, an “isolated” or “purified” small molecule, nucleic acid molecule, polynucleotide, polypeptide, or protein (e.g., antibody or fragment thereof), is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally occurring state. Purified also defines a degree of sterility that is safe for administration to a human subject, e.g., lacking infectious or toxic agents.

By “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A small molecule is a low molecular weight compound of less than 1000 Daltons, less than 800 Daltons, or less than 500 Daltons.

In some embodiments, one or more of the compounds (e.g., Shp2 pathway inhibitors) described herein is formulated into a pharmaceutical composition. The methods of invention include administering a pharmaceutical composition comprising a Shp2 pathway inhibitor to a subject in need thereof. A pharmaceutical composition is a formulation containing a disclosed compound (e.g., Shp2 pathway inhibitor) in a form suitable for administration to a subject.

The following materials and methods were used to study Shp2 inhibition for the prevention/treatment of osteoporosis and osteolysis.

Transgenic Animals and Phenotypic Characterization

Transgenic mice that carry Ptpn11 floxed allele, Cathepsin K (Ctsk)-Cre (Nakamura T, et al. (2007) Cell 130(5):811-23), and Rosa26mTmG (R26mTG) (Muzumdar M D, et al. (2007) Genesis 45(9):593-605) reporter were maintained in a pathogen-free facility. All animal-related experimental procedures were approved by the Institutional Animal Care and Use Committee. To generate mice in which Shp2 was deleted only in cells that express cathepsin K, Ptpn11 floxed allele were bred to Ctsk-Cre mice, yielding offspring with the following genotypes and nomenclature: Ptpn11fl/+;Ctsk-Cre and Ptpn11fl/fl;Ctsk-Cre (hereafter Ctsk-Control and Ctsk-KO, respectively). To confirm the activity and specificity of Cre recombinase, Ctsk-Cre was also bred with R26mTG reporter mice to generate R26mTG;Ctsk-Cre mice.

X-Ray and Micro-CT Analysis

X-ray images of the entire skeleton were taken immediately after euthanasia with Faxitron imaging system (Wheeling, Ill.). μ-CT images of the femurs and knee joints were scanned with desktop microtomorgraphic imaging system (μ-CT40, Scanco Medical AG, CH) after fixation in 4% PFA. To estimate new bone mineralization rate, mice received ip injections of calcein (10 mg/kg) 7d and xylenol orange (90 mg/kg) 2 d before euthanasia. In this case, 8 μm-thick proximal tibia sections were cut and mounted unstained for evaluation of calcein and xylenol orange fluorescence.

Reagents

DMEM medium, fetal bovine serum, penicillin, and streptomycin were purchased from Invitrogen Inc (N.Y), soluble RANKL and murine M-CSF were purchased from Preprotech (Rocky Hill, N.J.) and R&D Systems (Minneapolis, Minn.). Tartrate resistant acid phosphatase (TRAP) staining kits and cell viability/proliferation kits were purchased from Sigma-Aldrich (St. Louis, Mo.) and Roche (Indianapolis, Ind.), respectively. Monoclonal antibodies against phospho(p)-tyrosine (4G10) were from EMD Millipore (Billerica, Mass.). Polyclonal antibodies against phospho(p)-Erk1/2, Erk2, p-Akt(Ser473), and Akt were from Cell Signaling (Danvers, Mass.), and polyclonal antibodies against Shp2, c-Fos, and Nfatc1 were from Santa Cruz Biotechnology (Santa Cruz, Calif.). Shp2 inhibitor NSC-87877 (NSC) (Chen L, et al. (2006) Mol Pharmacol 70(2):562-70) was purchased from TOCRIS Bioscience (Minneapolis, Minn.) and Bisphosphonates (BP) from Sigma-Aldrich (St. Louis, Mo.).

Bone Marrow Cell Isolation, Culture, and Osteoclastogenic Differentiation

Whole bone marrow (BM) was flushed from the explanted femurs and tibiae of euthanized mice with DMEM (Invitrogen, N.Y), and centrifuged at 1,500 rpm for 5 minutes. The pelletized cells were resuspended in red blood cell (RBC) lysis buffer, re-centrifuged at 1,500 rpm for 5 minutes, and then cultured in macrophage medium (DMEM supplemented with 10% FBS, 1% penicillin/streptomycin, and 10 ng/mL M-CSF) at 37° C. and 5% CO₂ for 3 days (Tanaka S, et al. (1993) J Clin Invest 91(1):257-63) in either 100 mm tissue culture dishes, 4-well or 8-well chamber slides, or 48-well or 96-well tissue culture plates. The medium was changed every other day. To induce osteoclastogenic differentiation, BM cells cultured for 3 days in macrophage medium were further cultured for 5-7 days in osteoclast differentiation medium (DMEM supplemented with 10% FCS, 10 ng/mL M-CSF, and 100 ng/mL RANKL) (McHugh K P, et al. (2000) J Clin Invest 105(4):433-40; Zou W, et al. (2008) Mol Cell 31(3):422-31). These BM-derived osteoclasts were primarily used for the various assays in this study.

Tartrate Resistant Acid Phosphatase (TRAP) Staining and Pit Assay

TRAP staining was used to identify multinucleated osteoclasts in osteoclastogenic cell cultures or tibia section. Briefly, tibia from control and Shp2 mutant mice were fixed in 4% paraformaldehyde overnight and then decalcified in EDTA solution (14%) till bone tissue was soft. The specimens were then dehydrated according standard procedures and embedded for sectioning. TRAP⁺ multinucleated osteoclasts (>3 nuclei) were quantified with the use of a Nikon digital microscope. Osteoclast-mediated bone resorptive activity was quantified using the pit assay (Xu D, et al. (2004) Biochemical Journal 383(Pt 2):219-225). Ethanol-sterilized slices of bovine bone were disposed in individual wells of a 48 well/plate with 1 mL osteoclast culture medium containing 2.5×10⁴ BM macrophages (BMM) per well. After 8 to 10 days, all cellular components were removed from bone slices with a brief (10 minute) treatment with 5% sodium hypochlorite. The bone slices were then washed with phosphate buffered saline (PBS) and osteoclast lacunae were revealed via staining with 0.1% Toluidine blue.

Cell Proliferation and Viability Assays

To measure the effect of Shp2 deletion on the proliferation and viability of osteoclasts, BM cells from Control and Shp2 KO mice were cultured in DMEM supplemented with M-CSF, RANKL, or both as the indicated doses. WST-1 solution was then added ( 1/10, v/v) 2 hours before measuring optical density on a spectrophotometer at 420 nm (Cook J A, et al. (1989) Anal Biochem 179(1):1-7). The effects of Shp2 inhibition by NSC and BP treatments on cell proliferation and viability were determined using a similar approach.

Quantitative RT-PCR (q-PCR) Assays

Total RNA was extracted from BM osteoclast cultures of Control and Shp2 KO mice using the RNeasy mini kit (Qiagen, Valencia, Calif.). cDNA was synthesized using 1 ug total RNA with iSCRIPT™ cDNA Synthesis Kit (Bio-Rad, Hercules, Calif.) and q-PCR was performed with RT²SYBR® Green qPCR kit (Bio-Rad) on a CFX384 real-time PCR machine. Gene expressions in OC were normalized to GAPDH levels before fold increases or decreases were calculated between Ctsk-KO and Ctsk-Control mice.

Cell Signaling Study and Protein Preparation

To examine the molecular mechanism of Shp2 in M-CSF and RANKL signaling, bone marrow-derived osteoclasts were deprived of growth factors for 14 hours in DMEM containing 0.2% FBS. The starved cells were then stimulated with M-CSF (30 ng/mL) or RANKL (100 ng/mL). After stimulation, cells were lysed at various time points in NP-40 lysis buffer (0.5% NP40, 150 mM NaCl, 1 mM EDTA, 50 mM Tris [pH 7.4]) supplemented with a protease inhibitor cocktail (1 mM phenylmethanesulfonyl fluoride (PMSF), 10 mg/mL aprotinin, 0.5 mg/mL antipain, and 0.5 mg/mL pepstatin). Total cell lysates were separated from debris by centrifugation at 14,000 rpm for 10 minutes and protein concentrations in individual supernatants were determined by the BCA method on a Nanodrop-2000 spectrophotometer.

Western Blot Analysis

For immunoblotting, cell lysates (30-50 ug) were resolved by SDS-PAGE, transferred to polyvinylidene fluoride (PVDF) membranes, and incubated with primary antibodies for 2 hours or overnight at 4° C. (according to the manufacturer's instructions). After a one-hour wash in tris-buffered saline and TWEEN™ (TBST) (1× tris-buffered saline (TBS) with 0.1% Tween-20), the membranes were incubated with horseradish peroxidase (HRP-conjugated secondary antibodies. Detection of the proteins of interest was done by enhanced chemiluminescence (Amersham).

Fluorescent Microscopy Imaging

To visualize the morphology of osteoclast, cells cultured in slide chambers were washed in phosphate buffered saline (PBS) twice. After a quick fixation in 4% paraformaldehyde for 10 minutes, slides were rinsed with PBS again and mounted with Vectshield mounting medium containing 4′,6-diamidino-2-phenylindole (DAPI). These slides were immediately analyzed under a Nikon digital fluorescence microscope.

Cell Infection and Gene Over-Expression

pMX-IRES-GFP-based constructs harboring Nfatc1 were described previously (Takayanagi H, et al. (2002) Dev Cell 3(6):889-901). To generate BMM cells expressing GFP or Nfatc1/GFP, Plat-E cells were transiently transfected with pMX constructs via Effectene (Qiagen, Valencia, Calif.) per the manufacturer's instructions. Viruses were collected 48 hours post-transfection and used to infect BM cells seeded the previous day in the presence of M-CSF (20 ng/mL) and polybrene (4 ug/mL). Infected cells were then expanded and used later in vitro for osteoclastogenesis assays.

Statistical Analysis

Statistical differences between groups were evaluated by student t and x2 tests. p values less than 0.05 were considered significant. Analyses were performed using Excel (Microsoft, Redmond, Wash.) and Prism 3.0 (GraphPad, San Diego, Calif.). Data are presented as mean± standard deviation (SD).

The invention will be further described in the following examples, which do not limit the scope of the invention described in the claims.

### Example 1

### Mice Lacking Shp2 in Cathepsin K-Expressing Cells are Osteopetrotic

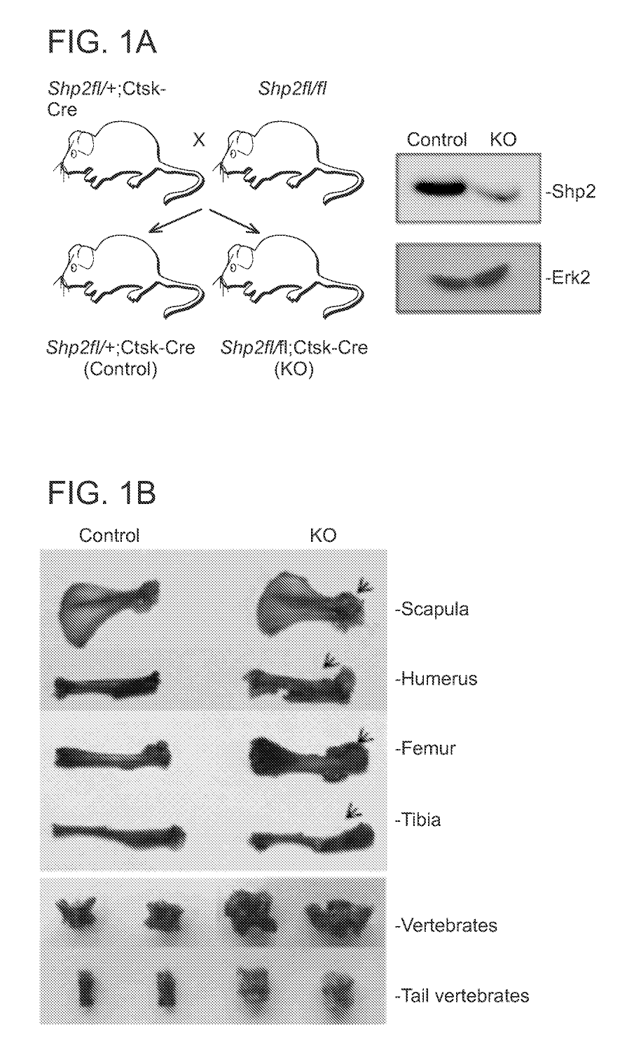

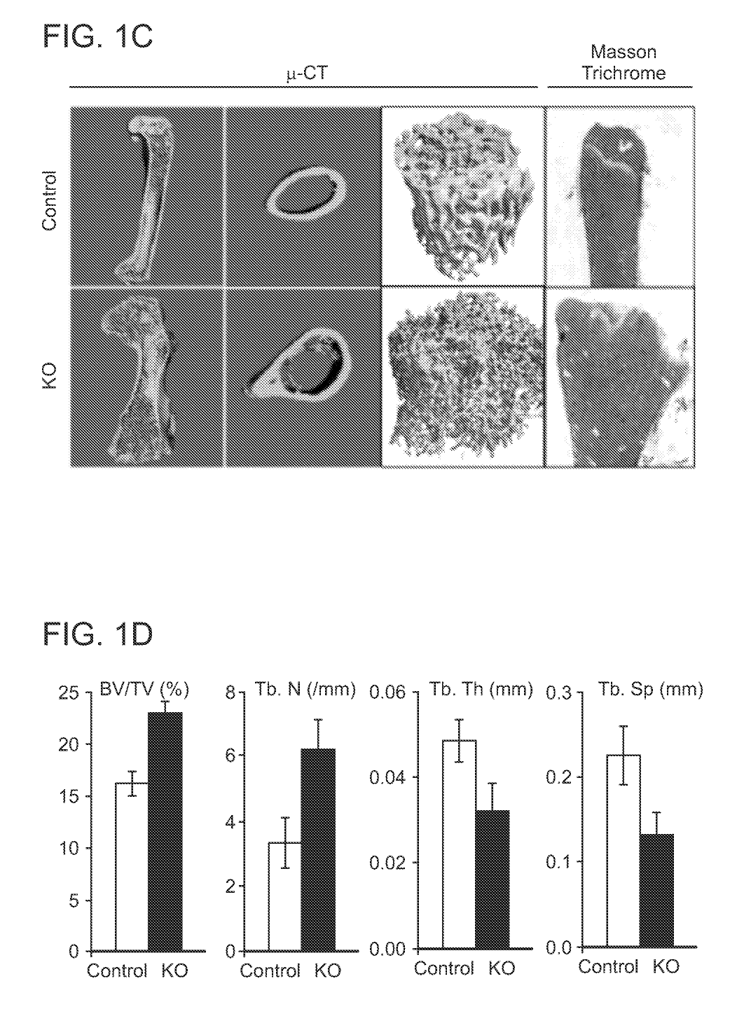

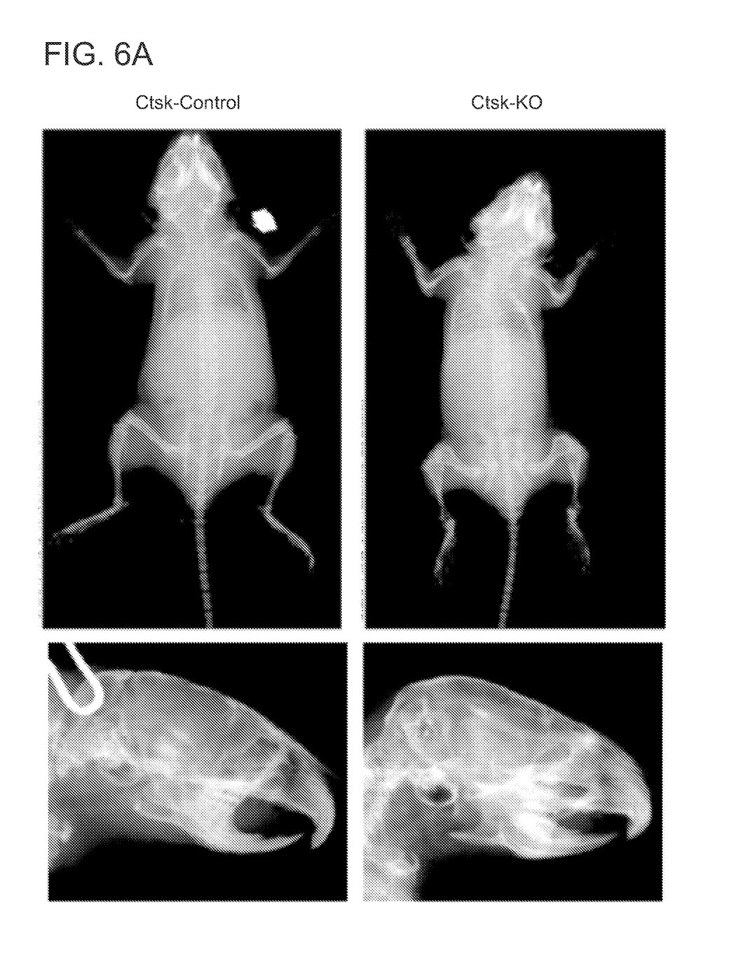

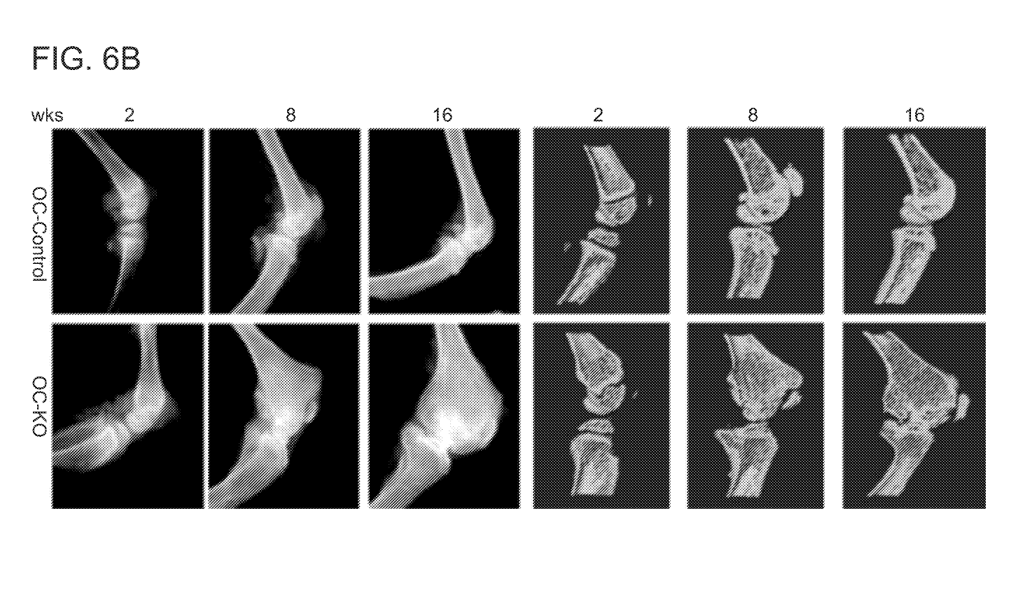

Control and KO mice were bred as described (FIG. 1A) and born at the expected Mendelian ratios. Western blot analysis showed that Ctsk-Cre mediated an efficient Shp2 deletion in osteoclasts derived from bone marrow cells from KO mice compared to Controls. Both Control and KO mice appeared normal in the first 3 weeks post-birth; but subsequently, Ctsk-KO developed several skeletal phenotypes, including short stature, increased bone mineral density (BMD), and scoliosis. Gross images showed that KO mice, compared to Controls, have short and widened femurs, tibiae, and humeri. Vertebrates and scapula were also affected (FIG. 1B). μCT imaging revealed markedly increased volumetric density (BV/TV) and trabecuale (Tb, N) in the KO mice compared to the Controls, but decreased trabecular thickness (Tb, Th) and spacing (Tb, Sp) (FIGS. 1C-D). Close observation demonstrate that both KO and Control mice had normal tooth eruption (FIG. 6). These data together suggest that Shp2 in osteoclasts significantly influences skeletal development and remodeling.

### Example 2

### Shp2 Deficiency Impairs Osteoclastogenesis In Vivo and Ex Vivo

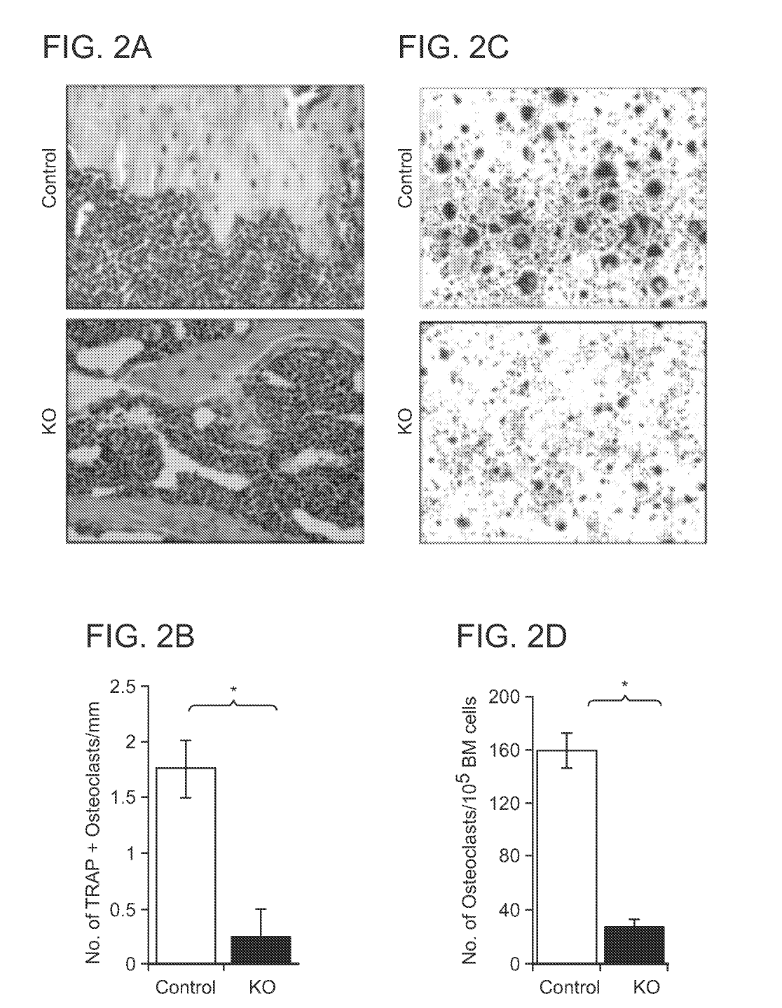

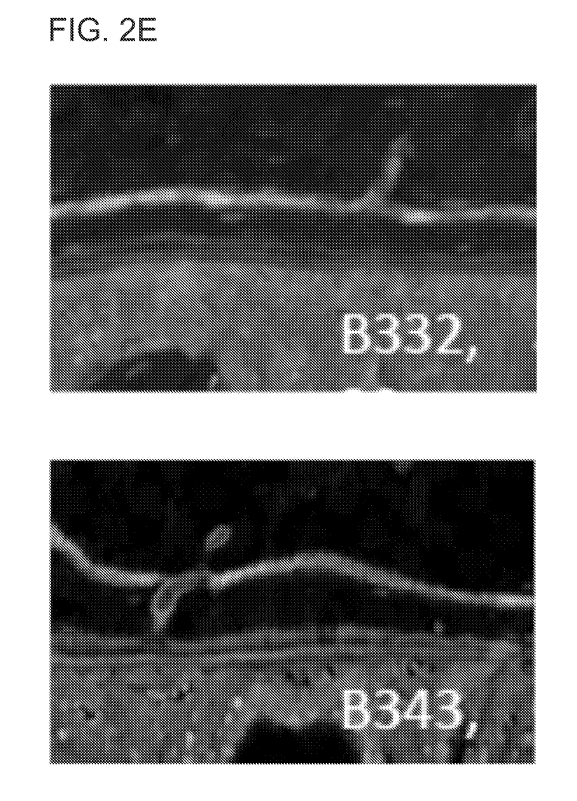

To further understand the cellular and molecular mechanisms of Shp2 in the above skeletal disease, the existence of osteoclasts in proximal tibia sections was visualized by TRAP staining and counter staining with Hematoxylin. TRAP-positive multinucleated osteoclasts were readily seen on the surface of trabecular bones in the metaphyseal regions of bones from Control mice, while TRAP-positive cells were substantially reduced in similar bone regions of KO mice (FIG. 2A). Histomorphometry analysis revealed that the number of osteoclasts per mm trabecular bone surface significantly decreased in KO mice compared to that of Control (FIG. 2B). This study provided the first set of genetic evidence demonstrating that Shp2 directly affects bone mass and micro-architecture, most likely through modulation of osteoclastogenesis and bone remodeling.

Osteoclastogenesis in vivo can be recapitulated ex vivo by culturing bone marrow cells in the presence of M-CSF and RANKL (Teitelbaum S L, et al. (2003) Nat Rev Genet 4(8):638-49; Tolar J, et al. (2004) N Engl J Med 351(27):2839-49). Experiments were carried out to test whether the blockade of osteoclast formation in Shp2 mutant mice can be mimicked in the above ex vivo system. Consistent with the observations in vivo, marrow cells from Shp2 KO mice indeed failed to generate multinucleated TRAP+ osteoclasts (FIGS. 2C-D). The data clearly demonstrate that Shp2 is required for M-CSF− and RANKL-evoked osteoclast development. These osteoclast culture experiments not only demonstrated that the phenotypes in KO mice can be recapitulated ex vivo, but also proved that Shp2 function during osteoclastogenesis is cell-autonomous.

### Example 3

### Shp2 Regulates Osteoclastogenesis by Promoting Preosteoclast Fusion

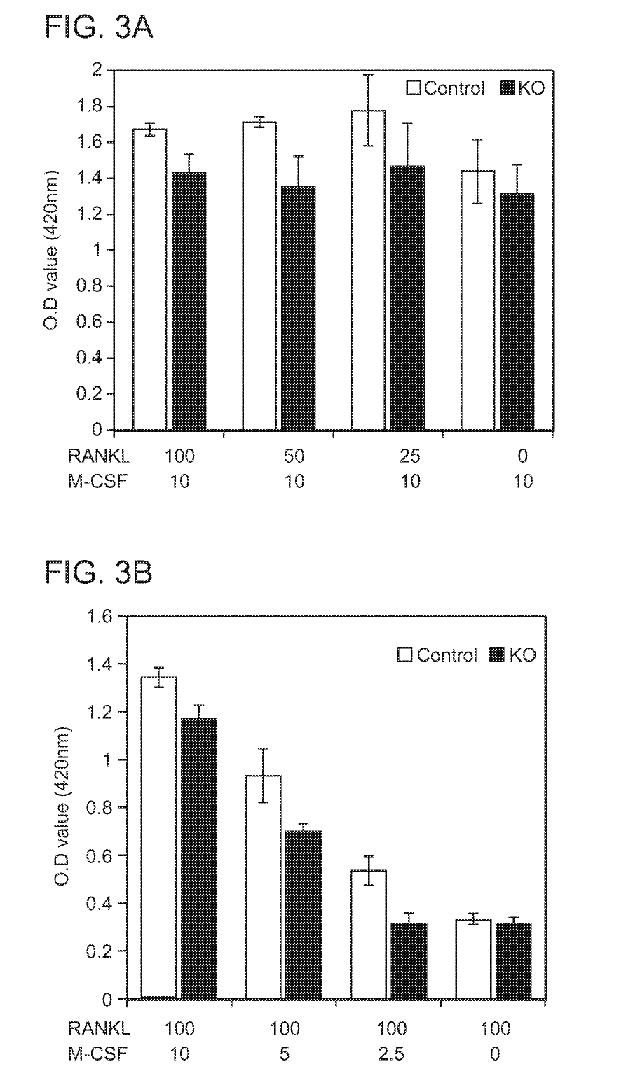

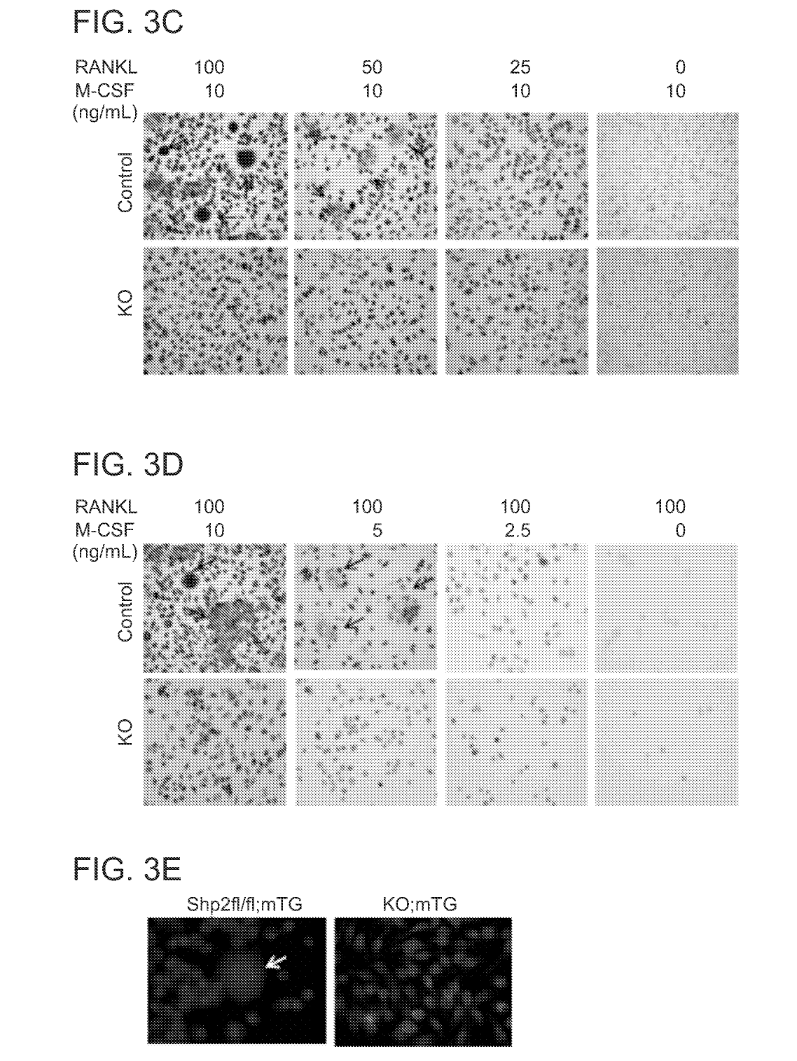

Having established that Shp2 is necessary for mature osteoclast formation, experiments were carried to out to test whether Shp2 deficiency influenced the viability or differentiation of osteoclast lineage. BM cells from Control and KO mice were cultured in medium containing a constant dose of M-CSF and variable amounts of RANKL, or conversely, with a constant dose of RANKL and variable amounts of M-CSF. Viability and proliferation of osteoclast precursors was evaluated with the WST-1 test, while differentiation was assessed via TRAP staining of multinucleated giant cells. The WST-1 results demonstrated that the proliferation and viability of osteoclast lineage cells from both Control and KO mice were comparable in the presence of optimal dose of M-CSF regardless of the RANKL dosing (FIGS. 3A and C). However, both proliferation and viability of osteoclast lineage cells from Control and KO mice declined when doses of M-CSF decreased, even in the presence of an optimal dose of RANKL (FIGS. 3B and D). These results suggest that the M-CSF-evoked signals primarily maintain cell proliferation and viability. Surprisingly, when similar cell populations were cultured in media containing an optimal dose of M-CSF and increased doses of RANKL, BM cells from Control mice formed increased numbers of both TRAP positive pre-osteoclasts and giant multinucleated osteoclasts (FIG. 3C), while BM cells from KO mice formed only TRAP positive pre-osteosteclasts and few if any fused multinucleated osteoclasts (FIG. 3D).

The above observation was further supported by a fluorescent protein-based reporter study. BM cells from Control and KO mice carrying mTG reporter were cultured in osteoclast differentiation medium. After inducing the expression of Ctsk-Cre, BM-derived pre-osteoclasts from KO mice changed their color from Tomato Red to GFP green. These green and Shp2 deficient pre-osteoclasts, consistent with the observation by TRAP assays, described above, failed to form any giant multinucleated osteoclasts. However, in the absence of Cre expression, these cells did not switch their color and still expressed Shp2. Importantly, these cells were able to form multinucleated osteoclasts (FIG. 3E). Taken together, this data supports that Shp2 regulates osteoclast formation by promoting preosteoclast fusion via a RANK dependent signaling pathway(s).

### Example 4

### Shp2 Regulates Pre-Osteoclast Fusion by Influencing NFATc1 Expression

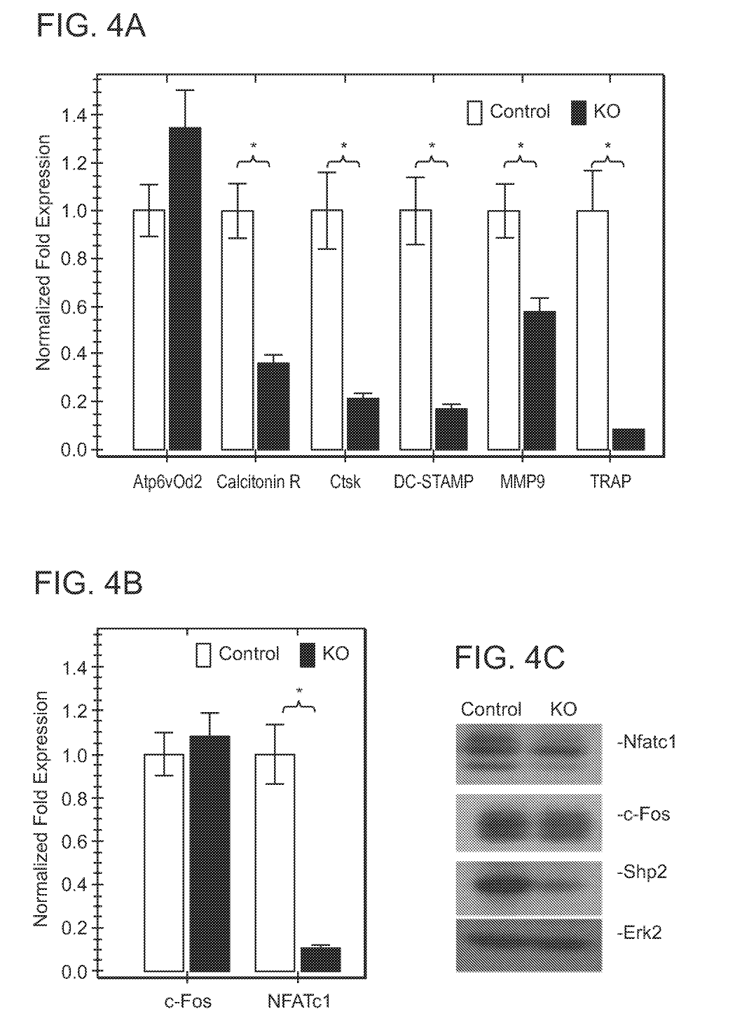

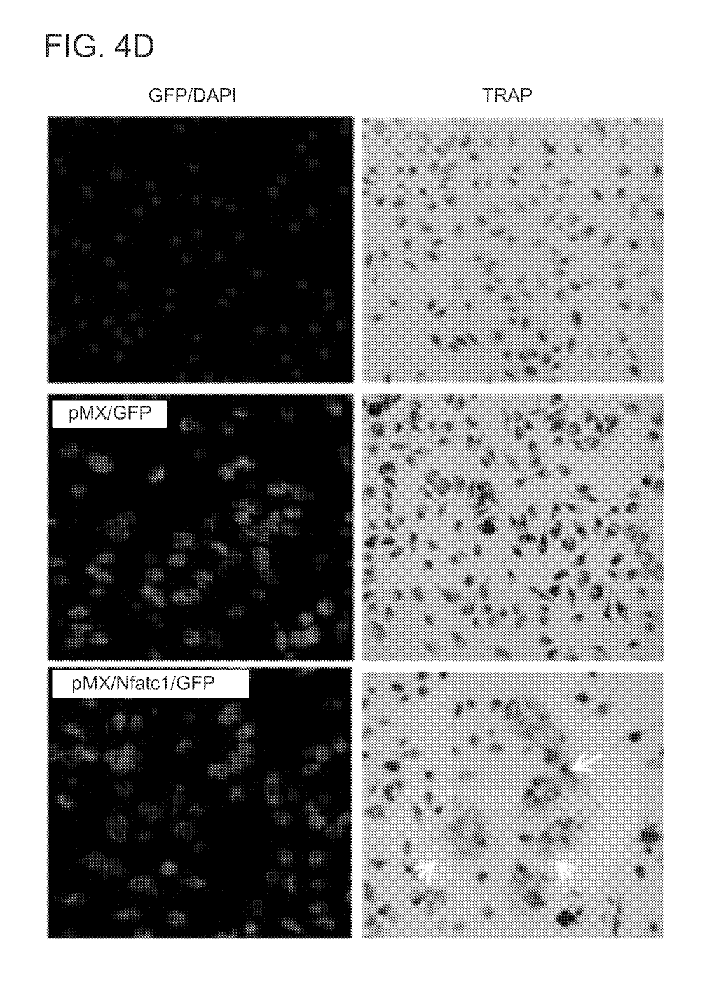

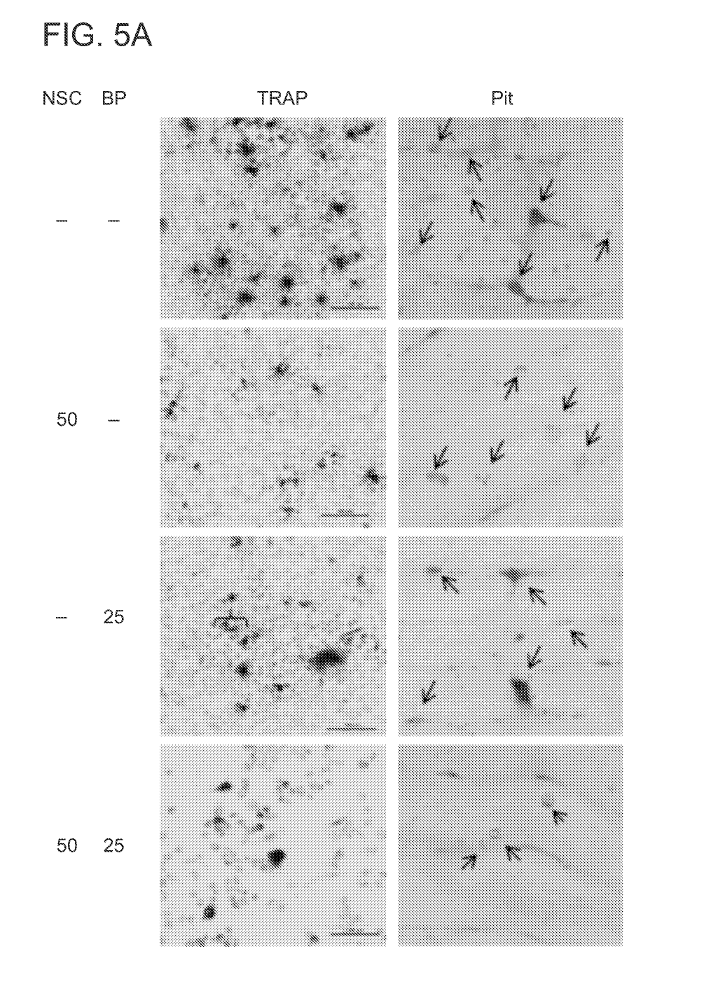

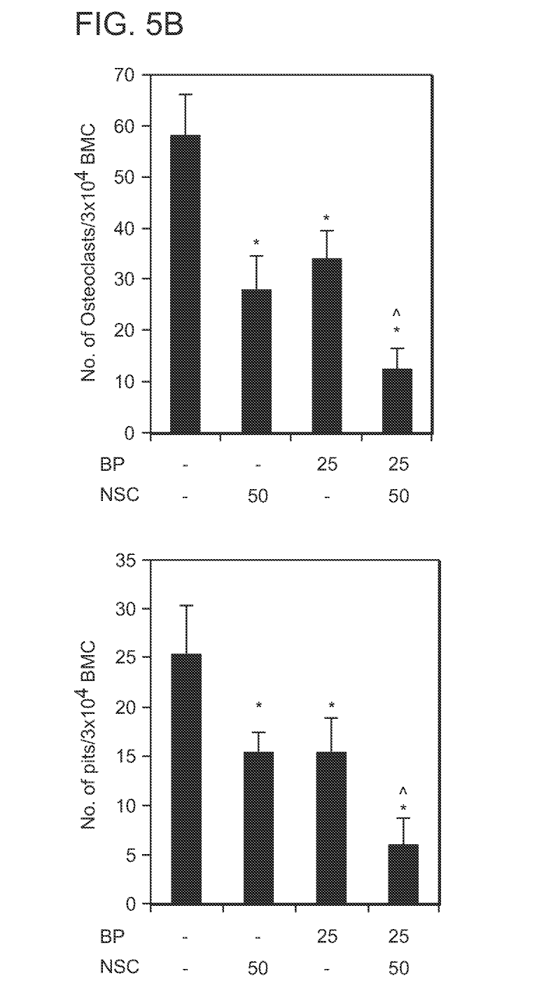

To explore how Shp2 regulates pre-osteoclast fusion, a q-PCR analysis for the expression of genes that are known to be involved in osteoclastogenesis was performed. BM cells from Control and KO mice were cultured in macrophage medium for 3 days and then switched to osteoclast medium for another 5 days. The cells were lysed and total RNA was extracted using a Qiagen RNeasy kit. Expression of osteoclast marker genes (Asagiri M, et al. (2007) Bone 40(2):251-64; Takayanagi H, et al. (2002) Dev Cell 3(6):889-901), including Atp6v0d2, Calcitonin receptor, Ctsk, DC-STAMP, Mmp9, Trap, c-Fos, and Nfatc1, were examined. The expression of terminal differentiation genes, such as Ctsk, Mmp9, Trap and Calcitonin receptor, were down-regulated in OC cultures from KO mice (FIGS. 4A-B); this is consistent with the observations that osteoclast terminal differentiation was impaired as a consequence of Shp2 deletion. However, it was surprising that, following M-CSF and RANKL induction, the expression of c-Fos and Atp6v0d2 remained unchanged while that of Nfatc1 and DC-STAMP decreased markedly in OC culture from KO mice (FIGS. 4A-B). This is inconsistent with previous reports that Atp6v0d2 and DC-STAMP, both critical for osteoclast fusion, are similarly regulated by Nfatc1 (Kim K, et al. (2008) Molecular endocrinology (Baltimore, Md.) 22(1):176-185) and that c-Fos and Nfatc1 are equally responsible for relaying RANK signaling (Matsuo K, et al. (2004) Journal of Biological Chemistry 279(25):26475-26480). To validate the above q-PCR results at a protein level, Western blots were conducted to determine protein levels of Nfatc1 and c-Fos, and indeed Nfatc1 levels markedly decreased in OC culture from KO mice but c-Fos levels remained the same in OC culture from both Shp2 KO and Control mice (FIG. 4C). These results indicate that Shp2 promotes pre-osteoclast fusion at a molecular level primarily by upregulating Nfatc1 expression.

Experiments were performed to test whether over-expression of Nfatc1 in Shp2-deficient BM cells rescues the fusion defects of pre-osteoclast from KO mice. IRES/GFP (control) or Nfatc1/IRES/GFP) were over-expressed in Shp2-deficient BM cells through retroviral infections (FIG. 4D). The fusion of Shp2-deficient pre-osteoclasts was rescued by exogenous Nfatc1 expression. Therefore, these experiments uncovered an important role of Shp2 in regulating Nfatc1 expression and pre-osteoclast fusion under RANK signaling pathway.

### Example 5

### Pharmacological Inhibition of Shp2 Synergizes with the Effect of BP on Osteoclast-Mediated Bone Resorption

Bisphosphonates (BP) are routinely used in clinic to treat osteoporosis and osteolysis by inducing osteoclast death. However, BP have been reported to alter bone mechanical properties and lead to atypical bone fracture. There is a need for an alternative approach that can reduce the dosage of BP may improve the outcomes of OP and osteolysis treatment. Given that Shp2 deficiency inhibits pre-osteoclast fusion (as described in the examples above), experiments were performed to test whether the pharmacological inhibition of Shp2 can serve as an alternative approach to lower BP doses without compromising BP inhibition of osteoclast-mediated bone resorption. BM-derived pre-osteoclast cultures were either untreated or treated with BP, Shp2 inhibitor (NSC), and combinations of these two compounds as indicated (FIGS. 4A-B). Drug untreated group showed robust formation of TRAP+ multinucleated osteoclasts. These cells, when cultured on dentin slices, formed nice pits, indicating that they are capable of resorbing bone matrix. Surprisingly, when these cells were either treated with BP or NSF, the number of osteoclasts and their ability to resorb bone matrix were inhibited. Moreover, there was a strong synergistic effect between these two drugs in reducing osteoclast formation as well as its bone resorptive function (FIGS. 4A-B). Therefore, modulation of Shp2 activity, alone or in combination with BP, is useful in the development of a better approach to inhibit osteoclast-mediated bone resorption and osteolysis and a better treatment for osteoporosis and other osteolytic diseases.

### Example 6

### Identification of Shp2 Inhibitory Compounds

Osteoclast precursor cells are cultured as described above. To induce osteoclastogenic differentiation, osteoclast precursor cells cultured for 3 days in tissue culture medium are further cultured for 5-7 days in osteoclast differentiation medium (DMEM supplemented with 10% FCS, 10 ng/mL M-CSF, and 100 ng/mL RANKL) (McHugh K P, et al. (2000) J Clin Invest 105(4):433-40; Zou W, et al. (2008) Mol Cell 31(3):422-31) in the presence or absence of a candidate compound or molecule. Assays as described in detail above are performed to determine the effect of the candidate compound or molecule on the Shp2 pathway in this cell culture system. For example, assays are performed to measure the level of Shp2 activity the osteoclast precursor cells. For example, the enzymatic activity of Shp2 is measured with a pNPP phosphatase assay in which pNPP (4-nitrophenyl phosphate) servers as a colorimetric substrate, and a water soluble yellow product with a strong absorption at 405 nm is measured with an ELISA reader. A decrease in the level of Shp2 activity in the presence of said compound compared to that in the absence of said compound indicates that said compound inhibits osteoclastogenesis. Alternatively or in addition to Shp2 activity level, the number of osteoclasts in the culture is determined, and a decrease in the number of osteoclasts in the presence of said compound compared to that in the absence of said compound indicates that said compound inhibits osteoclastogenesis.

Other methods of screening involve further testing a candidate Shp2 inhibitory compound or molecule in the osteoclast culture system and measuring the number or density of mature osteoclasts after culturing precursors in the presence and absence of the candidate compound. A decrease in the number or density of osteoclasts in the presence of the compound compared to that in its absence indicates that the compound inhibits osteoclastogenesis.

Blockade of osteoclast maturation or formation demonstrates efficacy of the candidate compound or molecule in treating or preventing osteoporosis or osteolysis. The candidate compound or molecule is used in the methods and compositions described herein. Compound and molecule libraries used in the screening include chemicals of known biological action, chemical compounds with unknown actions, and natural compound libraries. These libraries are available as non-commercial collections from the Molecular Libraries Program at the National Institute of Health and as commercial products from Sigma and ChemBridge Corporation. These screens identify novel Shp2 pathway inhibitors that can be applied for treating osteoporosis and osteolysis, e.g., in the methods above.

### Example 7

### Shp2 Pathway Inhibitors Improve Bone Ongrowth and Fixation of Orthopedic Implants

Orthopedic implants, such as hip and knee replacements, frequently fail due to the lack of early and stable fixation of the implant to the surrounding bone. In order for stable fixation of the implant to occur, new bone growth and ongrowth, a process in which bone grows onto or in contact with an implant surface, must take place during the healing process after surgery. The healing process involves new bone formation mediated by osteoblasts and bone resorption mediated by osteoclasts. Alendronate (a bisphosphonate compound) has been shown to increase bone ongrowth and shear strength (Jensen, T. B., et al. J. Orthopaedic Res. 2007, 772-778). However, bisphosphonates have a long half-life and thus interfere with long-term bone remodeling at the site of the implant.

A Shp2 pathway inhibitor is administered to a subject before, during, and/or after insertion of an orthopedic implant (e.g., a press-fit implant), such as a hip replacement, knee replacement, shoulder replacement, elbow replacement, finger joint, spine (vertebra) repair, or any other porous or porous-coated device into or onto which bone growth is desired. Administration is systemic (e.g., oral, i.v., or i.m.) or local (e.g., by injection or infusion). Optionally, the tissues, e.g. bone canal, into which the prosthesis is inserted is contacted with, e.g. by lavage, with a Shp2 inhibitor prior to insertion of the device into the bone. Monitoring of bone formation, ongrowth, and remodeling is performed by standard methods.

The Shp2 pathway inhibitor promotes bone ongrowth and shear strength at the site of the orthopedic implant. For example, Shp2 inhibitor is administered approximately one week prior to surgery. Surgery is then performed to insert the implant. Shp2 inhibitor is administered for approximately 6 weeks following the surgery or until the bone healing process is substantially complete. Administration of the inhibitor is stopped several weeks (e.g., about 6 weeks) after the insertion of the implant to allow further bone remodeling o occur. The cessation of administration of small molecule Shp2 inhibitors relieves the inhibition of osteoclasts and allows them to function in remodeling the bone at the site of the implant.

### Example 8

### Blockade of Shp2/Nfatc1 Signaling Pathway Inhibits Polyethylene and Titanium Particles-Induced Osteoclastogenic Gene Expression and Osteoclast-Mediated Bone Resorption

The effect of inhibition of Shp2 enzymatic activity on the expression of osteoclastogenic genes induced by polyethylene and titanium particles was tested Polyethylene (Pe) and titanium (Ti) are the two types of materials commonly used in orthopedic implants. Wear debris produced by polyethylene and titanium implants in orthopedic patients reportedly trigger local osteoclastogenesis and subsequently periprosthetic osteolysis. To test whether Shp2 inhibition can ameliorate wear-debris induced osteoclastogenesis in vitro, Pe particles (PeP) and Ti particles (TiP) were used in this study to induce osteoclastogenic differentiation of bone marrow-derived macrophages (BMM).

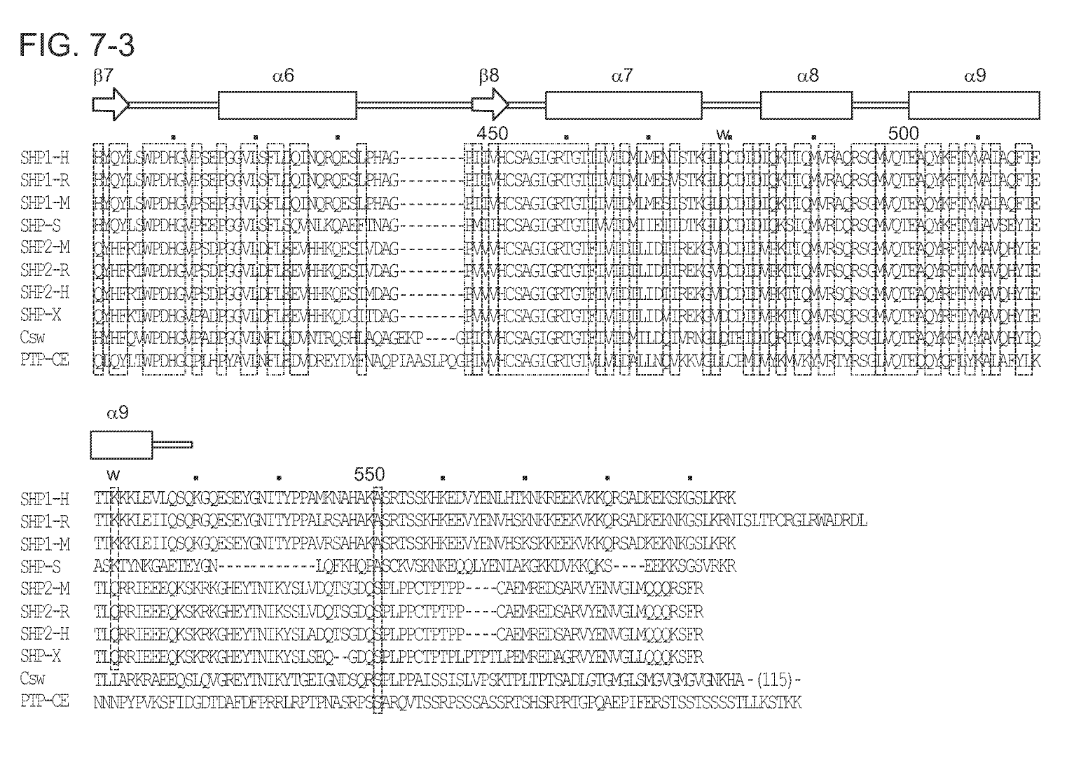

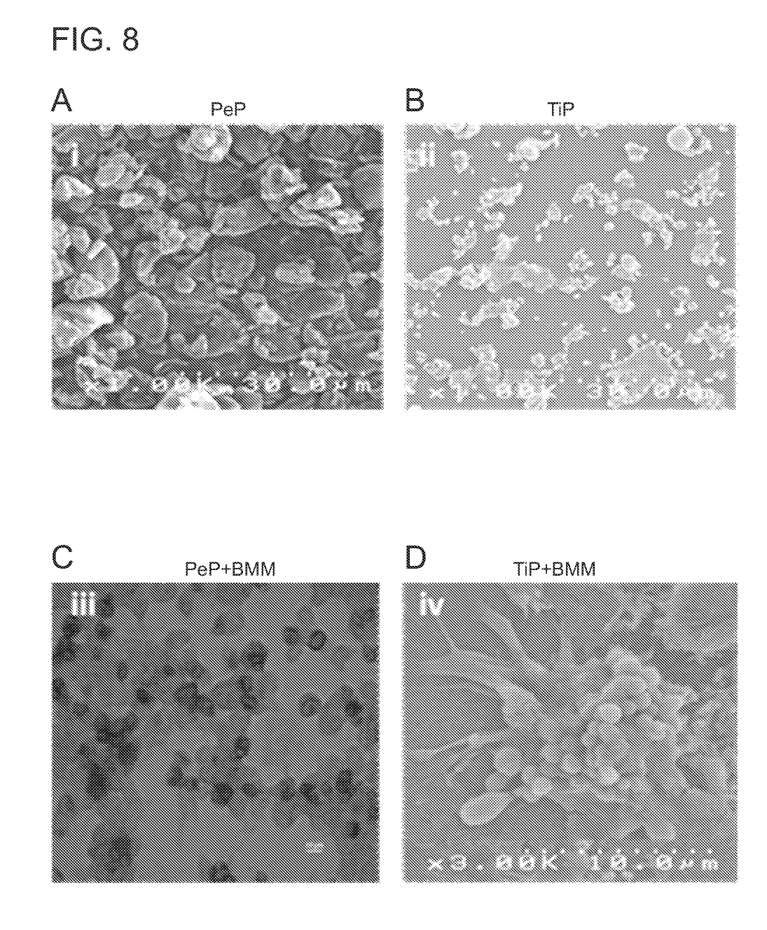

The average size of PeP and TiP is about 5-10 nm in diameter (FIGS. 8A-B), within the range of orthopedic implant debris size; these particles can be efficiently engulfed by BMM and trigger their osteoclastogenic differentiation (FIGS. 8C-D). 5×10⁵ BMM were cultured in BMM medium containing about 2×10⁶ of PeP or TiP in the presence or absence of Shp2 inhibitor NSC87877 (50 uM). BMM cultured in osteoclast media (DMEM supplemented with 10% FCS, 10 ng/mL M-CSF, and 100 ng/mL RANKL) were used as a positive control. Total RNA was extracted from BMM after 5 days of culture using RNeasy mini kit (Qiagen, Valencia, Calif.). cDNA was synthesized using 1 μg total RNA with iSCRIPT™ cDNA Synthesis Kit (Bio-Rad, Hercules, Calif.) and q-PCR was performed with RT2SYBR® Green qPCR kit (Bio-Rad) on a CFX384 real-time PCR machine. Osteoclastogenic gene expressions in BMM were measured and normalized to Glyceraldehyde 3-phosphate dehydrogenase (GAPDH); their levels were then compared between NSC87877 treated and untreated groups.

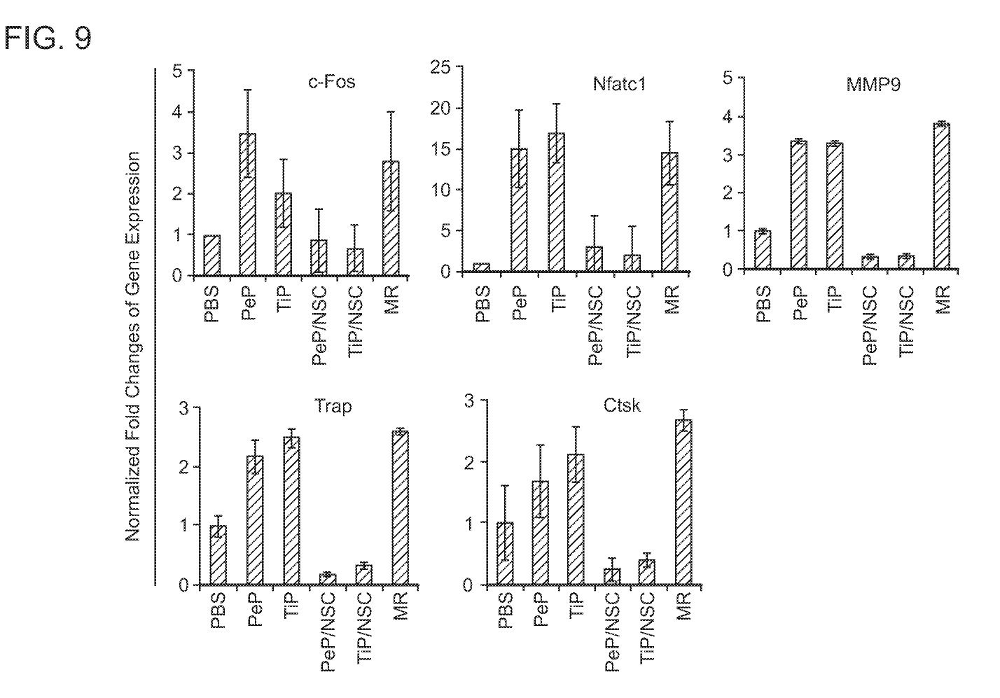

As shown in FIG. 9, PeP or TiP alone induced osteoclastogenic differentiation of BMM, supported by the increased expression of osteoclastogenic marker genes Nfatc2, c-Fos, MMP9, Trap and Ctsk. Surprisingly, NSC87877 treatment of BMM significantly inhibited the expression of these genes (FIG. 9), showing that blockade of Shp2-modulated signaling pathway(s) inhibits PeP and TiP-evoked osteoclastogenic differentiation.

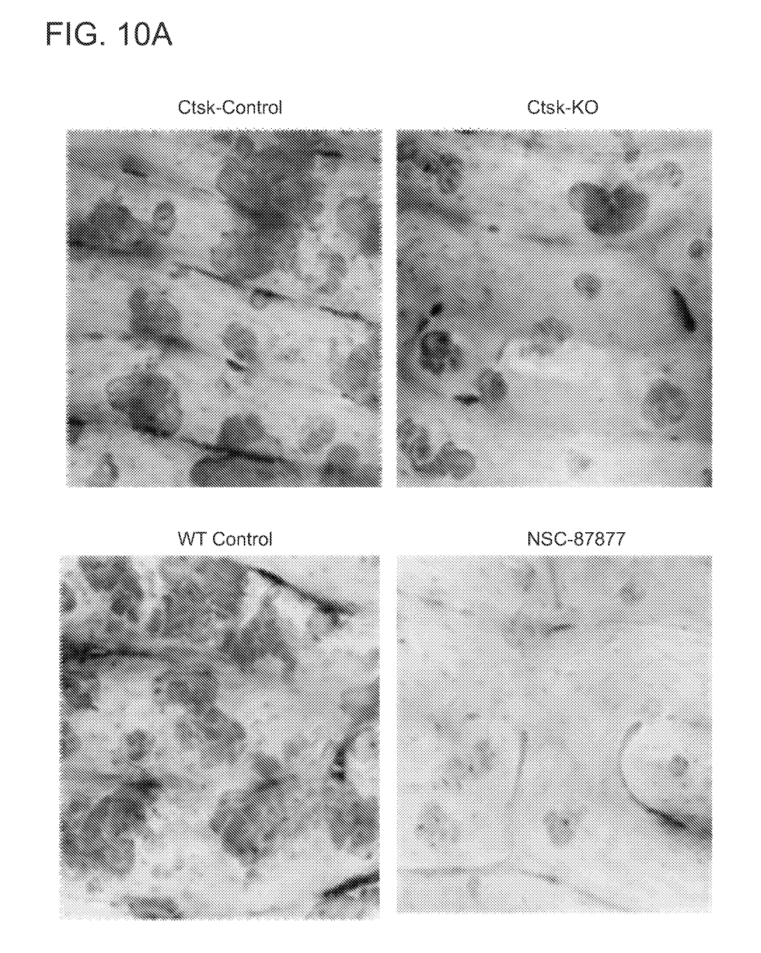

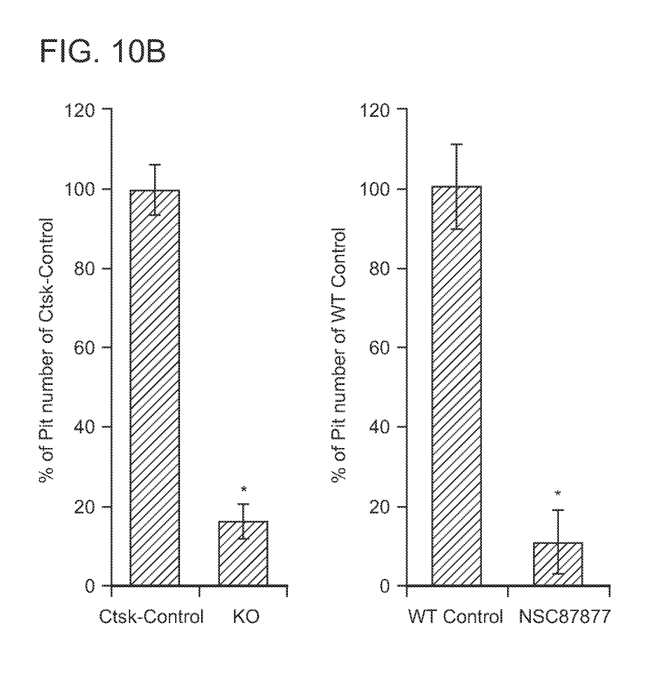

The effect of Shp2 inhibition on osteoclast-mediated bone resorption was also studied in order to further test whether pharmacological inhibition of Shp2 could serve as a treatment modality. BMM were cultured on dentin slices in osteoclast medium and in the presence or absence of Shp2 inhibitor NSC-87877 (50 μM) (FIGS. 10A-B). Osteoclasts from Ctsk-Control mice or from wild type mice treated with vehicle showed a robust formation of tulodin-stained pits on dentin surfaces, indicating that these osteoclasts were capable of resorbing bone matrix. Surprisingly, osteoclasts from Ctsk-KO mice or from wild type mice treated with NSC87877 formed only a few small pits, showing that Shp2 deletion or inhibition inhibited osteoclast bone resorptive activity (FIGS. 10A-B).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating or preventing osteolysis or osteoporosis, comprising identifying a subject comprising osteolysis or osteoporosis or comprising a risk for developing osteoporosis, and administering to said subject a Shp2 pathway inhibitor, wherein the Shp2 pathway inhibitor inhibits fusion of pre-osteoclasts.

2. The method of claim 1, wherein the Shp2 pathway inhibitor comprises a Shp2 inhibitor.

3. The method of claim 2, wherein the Shp2 inhibitor binds to the catalytic site of Shp2.

4. The method of claim 2, wherein the Shp2 inhibitor binds to an amino acid comprising the protein tyrosine phosphatase (PTP) domain of Shp2.

5. The method of claim 2, wherein the Shp2 inhibitor binds to one or more amino acids in human Shp2, wherein the one or more amino acids are selected from the group consisting of K280, Y279, N280, R362, H426, S460, A461, I463, G464, and R465, or an homologous residue thereof of a non-human Shp2.

6. The method of claim 2, wherein the Shp2 inhibitor inhibits an activity of human Shp2.

7. The method of claim 6, wherein the human Shp2 comprises the amino acid sequence of SEQ ID NO: 2.

8. The method of claim 2, wherein the Shp2 inhibitor prevents binding of Shp2 to a binding partner.

9. The method of claim 8, wherein the binding partner is Gab1 or Gab2.

10. The method of claim 2, wherein the Shp2 inhibitor comprises NSC87877, SPI-112, SPI-112Me, PHPS1, SHP2 inhibitor II-B08, C21, tautomycetin, TTN D-1,7-deshydroxypyrogallin-4-carboxylic acid (DCA), NSC-117199, 8Z,11Z-Feptadecadienoic acid, 14Z,17Z-tricosadienoic acid, caffeic acid, or 2-hydroxy-3-[(1-oxododecyl)oxy]propyl-β-d-glucopyranoside.

11. The method of claim 10, wherein the Shp2 inhibitor comprises NSC-87877.

12. The method of claim 1, wherein the Shp2 pathway inhibitor reduces an activity of or an expression level of Gab2, Nfatc1, Nfatc2, c-Fos, Calcitonin receptor, Ctsk, DC-STAMP, Mmp9, or Trap.

13. The method of claim 1, wherein the Shp2 pathway inhibitor reduces resorption of a bony tissue in the subject.

14. The method of claim 1, wherein identifying a subject with periprosthetic osteolysis or osteoporosis or at risk for osteoporosis comprises performing diagnostic imaging of the subject, wherein one or more of:
a) a zone of periprosthetic lucency greater than 2 mm on a radiograph;
b) a radiolucent lesion with well-defined borders on a computed tomograph; and
c) a well-defined lesion detected by magnetic resonance imaging
is indicative of a subject with periprosthetic osteolysis.

15. The method of claim 1, wherein identifying a subject comprising osteolysis or osteoporosis or comprising a risk for developing osteoporosis comprises measuring a bone density of a subject, wherein a bone density of the subject less than the average bone density of a normal healthy adult population is indicative of a subject comprising osteoporosis or comprising a risk for developing osteoporosis.

16. The method of claim 1, wherein a subject at risk for osteoporosis is identified by a subject having one or more risk factors for osteoporosis, wherein the one or more risk factors is selected from the group consisting of i) female and age of 65 or above, ii) male and age of 70 or above, iii) postmenopausal, age 65 or lower, and having one or more additional risk factors for osteoporosis, iv) male, age 50-70, and having one or more additional risk factors for osteoporosis, v) age 50 or above and having suffered a fracture, vi) long-term use of a medication associated with low bone density or bone loss, vii) long-term use of a corticosteroid, viii) a history of prostate cancer treatment or breast cancer treatment, ix) a history of diabetes, x) reduced calcium absorption in the gut, xi) prior gastric surgery, xii) caffeine intake, xiii) cigarette smoking, xiv) a family history of fracture or fragility, xv) physical inactivity, xvi) estrogen or thiazide use, xvii) alcohol consumption, xviii) a calcium, vitamin K, or vitamin D deficiency, xix) thyroid imbalance, xx) estrogen or testosterone deficiency, xxi) early menopause, xxii) anorexia nervosa, xxiii) rheumatoid arthritis, xxiv) significant loss of height, xxv) significant weight loss, and xxvi) low body mass index.

17. The method of claim 1, wherein administering comprises injecting, infusing, delivering endoscopically, implanting surgically, delivering arthroscopically, delivering with minimally invasive surgery, or delivering orally.

18. A pharmaceutical composition comprising a Shp2 pathway inhibitor.

19. The composition of claim 18, further comprising a bisphosphonate.

20. The composition of claim 19, wherein the bisphosphonate is selected from the group consisting of Aldronate, Etidronate, Clodronate, Tiludronate, Pamidronate, Neridronate, Olpadronate, Alendronate, Ibandronate, Risedronate, and Zoldronate.

21. The composition of claim 18, wherein the Shp2 pathway inhibitor comprises a Shp2 inhibitor.

22. The composition of claim 18, wherein the Shp2 inhibitor binds to one or more amino acids comprising the catalytic site of Shp2.

23. The composition of claim 21, wherein the Shp2 inhibitor binds to an amino acid comprising the protein tyrosine phosphatase (PTP) domain of Shp2.

24. The composition of claim 21, wherein the Shp2 inhibitor binds to one or more amino acids in human Shp2, wherein the one or more amino acids are selected from the group consisting of K280, Y279, N280, R362, K364, K366, W423, P424, D425, H426, G427, S460, A461, I463, G464, R465, and Q510, and an homologous residue thereof of a non-human Shp2.

25. The composition of claim 21, wherein the Shp2 inhibitor inhibits an activity of human Shp2.

26. The composition of claim 25, wherein the human Shp2 comprises the amino acid sequence of SEQ ID NO: 2.

27. The composition of claim 21, wherein the Shp2 inhibitor prevents binding of Shp2 to a binding partner.

28. The composition of claim 27, wherein the binding partner is Gab1 or Gab2.

29. The composition of claim 21, wherein the Shp2 inhibitor comprises NSC87877, SPI-112, SPI-112Me, PHPS1, SHP2 inhibitor II-B08, C21, tautomycetin, TTN D-1,7-deshydroxypyrogallin-4-carboxylic acid (DCA), NSC-117199, 8Z,11Z-Feptadecadienoic acid, 14Z,17Z-tricosadienoic acid, caffeic acid, or 2-hydroxy-3-[(1-oxododecyl)oxy]propyl-β-d-glucopyranoside.

30. The composition of claim 29, wherein the Shp2 inhibitor comprises NSC-87877.

31. The composition of claim 21, wherein the Shp2 pathway inhibitor comprises an inhibitor of a protein selected from the group consisting of Gab2, Nfatc1, Nfatc2, c-Fos, Calcitonin receptor, Ctsk, DC-STAMP, Mmp9, and Trap.

32. A device comprising an implant and a Shp2 pathway inhibitor, wherein the Shp2 pathway inhibitor is incorporated into, coupled to, or coated onto said implant.

33. The device of claim 32, wherein the implant comprises an orthopedic implant or a dental implant.

34. The device of claim 32, wherein the orthopedic implant is selected from the group consisting of bone screws, orthopedic pins, mechanical devices for the fixation and stabilization of an orthopedic fracture, bone cement, artificial joints, hip replacement joints, joint implants, hip replacements, knee replacements, elbow replacements, synthetic joints, synthetic cartilage, synthetic spin discs, bone plates, orthopedic nails, orthopedic rods, orthopedic rectangles, compression plates, shoulder replacements, bone wires, and prostheses.

35. A method of treating or preventing osteoporosis or osteolysis comprising administering to a subject the device of claim 32.

36. The method of claim 35, wherein said device is administered by injection, endoscopic delivery, minimally invasive surgery, arthroscopy, or surgical implantation.

37. A method for identifying a candidate compound for inhibiting osteoclastogenesis, said method comprising: (a) contacting a cell expressing an osteoclast precursor cell with a candidate compound and (b) measuring the level of Shp2 activity in said cell, wherein a decrease in the level of Shp2 activity in the presence of said compound compared to that in the absence of said compound indicates that said compound inhibits osteoclastogenesis.

38. A method for identifying a candidate compound for inhibiting osteoclastogenesis, said method comprising: (a) contacting a cell expressing an osteoclast precursor cell with a candidate compound in culture and (b) measuring the number of osteoclasts in the culture, wherein a decrease in the number of osteoclasts in the presence of said compound compared to that in the absence of said compound indicates that said compound inhibits osteoclastogenesis.

39. A method of improving fixation of an orthopedic implant, comprising administering a Shp2 pathway inhibitor to a subject before, concurrently with, and/or after insertion of said orthopedic implant.

40. The method of claim 39, comprising administering a Shp2 pathway inhibitor to a subject 5-10 days before insertion of the orthopedic implant.

41. The method of claim 39, comprising administering a therapeutically effective dosage of a Shp2 pathway inhibitor to a subject at the time of surgery and until bone healing is substantially complete, followed by gradually decreasing the dosage of the Shp2 pathway inhibitor, and cessation of the administration of the Shp2 pathway inhibitor.

42. The method of claim 39, wherein the administering comprises oral intake.

43-44. (canceled)
